(19) United States
(12) Patent Application Publication
(10) Pub. No.: US 2003/0083483 A1
(43) Pub. Date: May 1, 2003

(54) Molecular interaction sites of vimentin RNA and methods of modulating the same

(76) Inventors: David J. Ecker, Encinitas, CA (US); Richard Griffey, Vista, CA (US); Stanley T. Crooke, Carlsbad, CA (US); Ranga Sampath, San Diego, CA (US); Eric E. Swayze, Carlsbad, CA (US); Venkatraman Mohan, Plainsboro, NJ (US); Steven Hofstadler, Oceanside, CA (US); John McNeil, La Jolla, CA (US)

Correspondence Address: Paul K. Legaard, WOODCOCK WASHBURN LLP, 46th Floor, One Liberty Place, Philadelphia, PA, 19103, US

(21) Appl. No.: 10/135,017

(22) Filed: Apr. 24, 2002

Related U.S. Application Data
(63) Continuation-in-part of application No. 09/310,907, filed on May 12, 1999.

(51) Int. Cl. C07H 21/02; C12Q 1/68

(52) U.S. Cl. 536/23.1; 435/6

(57) ABSTRACT

Methods for the identification of compounds which modulate, either inhibit or stimulate, biomolecules are provided. Nucleic acids, especially RNAs are preferred substrates for such modulation. The present methods are particularly powerful in that they provide novel combinations of techniques which give rise to compounds, usually “small” organic compounds, which are highly potent modulators of RNA and other biomolecular activity. In accordance with preferred aspects of the invention, very large numbers of compounds may be tested essentially simultaneously to determine whether they are likely to interact with a molecular interaction site and modulate the activity of the biomolecule. Pharmaceuticals, veterinary drugs, agricultural chemicals, industrial chemicals, research chemicals and many other beneficial compounds may be identified in accordance with embodiments of this invention.

### CROSS-REFERENCE TO RELATED APPLICATIONS

This application is a continuation-in-part of U.S. Ser. No. 09/310,907 filed May 12, 1999, which is incorporated herein by reference in its entirety.

### FIELD OF THE INVENTION

The present invention relates to the identification of compounds which modulate, either inhibit or stimulate, biomolecules. Nucleic acids, especially RNA, are preferred substrates for such modulation and all such substrates are denominated “targets” for such action. The present methods are particularly powerful in that they provide novel combinations of techniques which give rise to compounds, usually “small” organic compounds, which are highly potent modulators of RNA and other biomolecular activity. Very large numbers of compounds may be tested in silico to determine whether they are likely to interact with a molecular interaction site and, hence, modulate the activity of the biomolecule. Pharmaceuticals, veterinary drugs, agricultural chemicals, industrial chemicals, research chemicals and many other beneficial compounds may be identified in accordance with embodiments of this invention. In particular, the present invention relates to identification of molecular interaction sites of vimentin.

### BACKGROUND OF THE INVENTION

Recent advances in genomics, molecular biology, and structural biology have highlighted how RNA molecules participate in or control many of the events required to express proteins in cells. Rather than function as simple intermediaries, RNA molecules actively regulate their own transcription from DNA, splice and edit mRNA molecules and tRNA molecules, synthesize peptide bonds in the ribosome, catalyze the migration of nascent proteins to the cell membrane, and provide fine control over the rate of translation of messages. RNA molecules can adopt a variety of unique structural motifs, which provide the framework required to perform these functions.

“Small” molecule therapeutics, which bind specifically to structured RNA molecules, are organic chemical molecules which are not polymers. “Small” molecule therapeutics include the most powerful naturally-occurring antibiotics. For example, the aminoglycoside and macrolide antibiotics are “small” molecules that bind to defined regions in ribosomal RNA (rRNA) structures and work, it is believed, by blocking conformational changes in the RNA required for protein synthesis. Changes in the conformation of RNA molecules have been shown to regulate rates of transcription and translation of mRNA molecules.

An additional opportunity in targeting RNA for drug discovery is that cells frequently create different mRNA molecules in different tissues that can be translated into identical proteins. Processes such as alternative splicing and alternative polyadenylation can create transcripts that are unique or enriched in particular tissues. This provides the opportunity to design drugs that bind to the region of RNA unique in a desired tissue, including tumors, and not affect protein expression in other tissues, or affect protein expression to a lesser extent, providing an additional level of drug specificity generally not achieved by therapeutic targeting of proteins.

RNA molecules or groups of related RNA molecules are believed by Applicants to have regulatory regions that are used by the cell to control synthesis of proteins. The cell is believed to exercise control over both the timing and the amount of protein that is synthesized by direct, specific interactions with mRNA. This notion is inconsistent with the impression obtained by reading the scientific literature on gene regulation, which is highly focused on transcription. The process of RNA maturation, transport, intracellular localization and translation are rich in RNA recognition sites that provide good opportunities for drug binding. The present invention is directed to finding these regions for RNA molecules in the human genome as well as in other animal genomes and prokaryotic genomes.

Combinatorial chemistry is a recent addition to the toolbox of chemists and represents a field of chemistry dealing with the synthesis of a large number of chemical entities. This is generally achieved by condensing a small number of reagents together in all combinations defined by a given reaction sequence. Advances in this area of chemistry include the use of chemical software tools and advanced computer hardware which has made it possible to consider possibilities for synthesis in orders of magnitude greater than the actual synthesis of the library compounds. The concept of “virtual library” is used to indicate a collection of candidate structures that would theoretically result from a combinatorial synthesis involving reactions of interest and reagents to effect those reactions. It is from this virtual library that compounds are selected to be actually synthesized.

Project Library (MDL Information Systems, Inc., San Leandro, Calif.) is said to be a desktop software system which supports combinatorial research efforts. (Practical Guide to Combinatorial Chemistry, A. W. Czarnik and S. H. DeWitt, eds., 1997, ACS, Washington, D.C.) The software is said to include an information-management module for the representation and search of building blocks, individual molecules, complete combinatorial libraries, and mixtures of molecules, and other modules for computational support for tracking mixture and discrete-compound libraries.

Molecular Diversity Manager (Tripos, Inc., St. Louis, Mo.) is said to be a suite of software modules for the creation, selection, and management of compound libraries. (Practical Guide to Combinatorial Chemistry, A. W. Czarnik and S. H. DeWitt, eds., 1997, ACS, Washington, D.C.) The LEGION and SELECTOR modules are said to be useful in creating libraries and characterizing molecules in terms of both 2-dimensional and 3-dimensional structural fingerprints, substituent parameters, topological indices, and physicochemical parameters.

Afferent Systems (San Francisco, Calif.) is said to offer combinatorial library software that creates virtual molecules for a database. It is said to do this by virtually reacting precursor molecules and selecting those that could be actually synthesized (Wilson, C&EN, Apr. 27, 1998, p.32).

While only Project Library and Molecular Diversity Manager are available commercially, these products do not provide facilities to efficiently track reagents and synthesis conditions employed for the introduction of fragments into the desired compounds being generated. Further, these products are unable to track mixtures of compounds that are generated by the introduction of multiple fragments by the use of multiple reagents. Therefore, it is desirable to have available methods for handling mixtures of compounds, as well as methods for the tracking of chemical reactions or transformations utilized in the synthesis of individual compounds and mixtures thereof.

The selection of compounds for synthesis and screening is a critical step in any drug discovery process. This is particularly true for combinatorial chemistry-based discovery strategies, where a very much larger number of compounds can be conceived than can be prepared in a reasonable time frame. Computational chemistry methods have been applied to find the “best” sets of compounds for screening. One strategy optimizes the chemical “diversity” in a library in order to increase the likelihood of finding a hit with biological activity in a screen against a macromolecular target of unknown structure.

Targeting nucleic acids has been recognized as a valid strategy for interference with biological pathways and the treatment of disease. In this regard, both deoxyribonucleic acids (DNA) and ribonucleic acids (RNA) have been the target of numerous therapeutic strategies. A wide variety of “small” molecules, oligomers and oligonucleotides have been shown to possess binding affinity for nucleic acids. The vast majority of experience in interfering with nucleic acid function has been via the specific binding of ligands to a particular base, base pair, and/or primary sequence of bases in the nucleic acid target. Some compounds have also demonstrated a composite specificity that arises from recognition and interactions with both the primary and secondary structural features of the nucleic acid, such as preferential binding to A-T base pairs in the DNA minor groove, with little or no binding to corresponding RNA sequences.

Exploiting the knowledge of the three-dimensional structure of biological targets is a promising strategy from a drug design and discovery standpoint. This has been demonstrated by the design and development of numerous drugs and drug candidates targeted to proteins involved in various pathophysiological pathways. While three dimensional structures of proteins have been widely determined by techniques such as X-ray crystallography, molecular modeling and NMR, nucleic acid targets have been difficult to study. The literature reveals few three dimensional structures of biologically active RNA, including a tRNA, said to have been determined via X-ray crystallography. Quigley, et al., Nucleic Acids Res., 1975, 2, 2329; and Moras, et al., Nature (London), 1980, 288, 669. The difficulties associated with proper crystallization and study of nucleic acids by X-ray methods along with the increasing number of biologically important small RNAs have increased the need for new structure determination and drug discovery strategies for such targets.

Many approaches to predicting RNA structure have been discussed in the scientific literature. Essentially, these involve sequencing and genomic analysis of nucleic acids, such as RNA, as a first step to establish the primary sequence structure and potential folded structures of the target. A second step entails definition of structural constraints such as base pairing and long range interactions among bases based on information derived from cross-linking, biochemical and genetic structure-function studies. This information, together with modeling and simulation software, has allowed scientists to predict three dimensional models of RNA and DNA. While such models may not be as powerful as X-ray crystal structures, they have been useful in ascertaining some structural features and structure-function relationships.

An understanding of the structural features of specific motifs in nucleic acids, especially hairpins, loops, helices and double helices, has been found to be useful in gaining molecular insights. For example, a hairpin motif comprising a double helical stem and a single-stranded loop is believed to be one of the simplest yet most important structural element in nucleic acids. Such hairpin structures are proposed to be nucleation sites and serve as major building blocks for the folded three dimensional structure of RNAs. Shen, et al., FASEB J., 1995, 9, 1023. Hairpins are also involved in specific interactions with a variety of proteins to regulate gene expression. Feng, et al., Nature, 1988, 334, 165, Witherell, et al., Prog. Nucleic Acids Res. Mol. Biol., 1991, 40, 185, and Phillipe, et al., J. Mol. Biol., 1990, 211, 415. Nucleic acid hairpin structures have therefore been widely studied by NMR, molecular modeling techniques such as constrained molecular dynamics and distance geometry (Cheong, et al., Nature, 1990, 346, 680 and Cain, et al., Nuc. Acids Res., 1995, 23, 2153), X-ray crystallography (Valegard, et al., Nature, 1994, 371, 623 and Chattopadhyaya, et al., Nature, 1988, 334, 175), and theoretical methods (Tung, Biophysical J., 1997, 72, 876, Erie, et al., Biopolymers, 1993, 33, 75, and Raghunathan, et al., Biochemistry, 1991, 30, 782).

The determination of potential three dimensional structures of nucleic acids and their attendant structural motifs affords insights into areas such as the study of catalysis by RNA, RNA-RNA interactions, RNA-nucleic acid interactions, RNA-protein interactions, and the recognition of small molecules by nucleic acids. Four general approaches to the generation of model three dimensional structures of RNA have been demonstrated in the literature. All of these employ sophisticated molecular modeling and computational algorithms for the simulation of folding and tertiary interactions within target nucleic acids, such as RNA. Westhof and Altman (Proc. Natl. Acad. Sci., 1994, 91, 5133) have described the generation of a three-dimensional working model of M1 RNA, the catalytic RNA subunit of RNase P from E. coli via an interactive computer modeling protocol.

Leveraging the significant body of work in the area of cryo-electron microscopy (cryo-EM) and biochemical studies on ribosomal RNAs, Mueller and Brimacombe (J. Mol. Biol., 1997, 271, 524) have constructed a three dimensional model of E. coli 16S Ribosomal RNA. A method to model nucleic acid hairpin motifs has been developed based on a set of reduced coordinates for describing nucleic acid structures and a sampling algorithm that equilibriates structures using Monte Carlo (MC) simulations (Tung, Biophysical J., 1997, 72, 876, incorporated herein by reference in its entirety).

MC-SYM is yet another approach to predicting the three dimensional structure of RNAs using a constraint-satisfaction method. Major, et al., Proc. Natl. Acad. Sci., 1993, 90, 9408. The MC-SYM program is an algorithm based on constraint satisfaction that searches conformational space for all models that satisfy query input constraints, and is described in, for example, Cedergren, et al., RNA Structure And Function, 1998, Cold Spring Harbor Lab. Press, p.37-75. Three dimensional structures of RNA are produced by that method by the stepwise addition of nucleotide having one or several different conformations to a growing oligonucleotide model.

Westhof and Altman (Proc. Natl. Acad. Sci., 1994, 91, 5133) have described the generation of a three-dimensional working model of Ml RNA, the catalytic RNA subunit of RNase P from E. coli via an interactive computer modeling protocol. This modeling protocol incorporated data from chemical and enzymatic protection experiments, phylogenetic analysis, studies of the activities of mutants and the kinetics of reactions catalyzed by the binding of substrate to M1 RNA. Modeling was performed for the most part as described in the literature. Westhof, et al., in “Theoretical Biochemistry and Molecular Biophysics,” Beveridge and Lavery (eds.), Adenine, NY, 1990, 399. In general, starting with the primary sequence of Ml RNA, the stem-loop structures and other elements of secondary structure were created. Subsequent assembly of these elements into a three dimensional structure using a computer graphics station and FRODO (Jones, J. Appl. Crystallogr., 1978, 11, 268) followed by refinement using NUCLIN-NUCLSQ afforded a RNA model that had correct geometries, the absence of bad contacts, and appropriate stereochemistry. The model so generated was found to be consistent with a large body of empirical data on M1 RNA and opens the door for hypotheses about the mechanism of action of RNase P. However, the models generated by this method are less well resolved that the structures determined via X-ray crystallography.

Mueller and Brimacombe (J. Mol. Biol., 1997, 271, 524) have constructed a three dimensional model of E. coli 16S ribosomal RNA using a modeling program called ERNA-3D. This program generates three dimensional structures such as A-form RNA helices and single-strand regions via the dynamic docking of single strands to fit electron density obtained from low resolution diffraction data. After helical elements have been defined and positioned in the model, the configurations of the single strand regions is adjusted, so as to satisfy any known biochemical constraints such as RNA-protein cross-linking and foot-printing data.

A method to model nucleic acid hairpin motifs has been developed based on a set of reduced coordinates for describing nucleic acid structures and a sampling algorithm that equilibriates structures using Monte Carlo (MC) simulations. Tung, Biophysical J., 1997, 72, 876, incorporated herein by reference. The stem region of a nucleic acid can be adequately modeled by using a canonical duplex formation. Using a set of reduced coordinates, an algorithm that is capable of generating structures of single stranded loops with a pair of fixed ends was created. This allows efficient structural sampling of the loop in conformational space. Combining this algorithm with a modified Metropolis Monte Carlo algorithm afforded a structure simulation package that simplifies the study of nucleic acid hairpin structures by computational means.

Knowledge and mastery of the foregoing techniques is assumed to be part of the ordinary skill in the art. There has been a long-felt need in the art to provide methods for improved determination of the three-dimensional structure of important regulatory and other elements in nucleic acids, especially RNA. It is also been greatly desired to achieve improved knowledge about the nature of interactions between ligands and potential ligands or nucleic acids, especially RNA. The present invention is directed towards satisfaction of these objectives.

The process of drug discovery is changing at a fast pace because of the rapid progress and evolution of a number of technologies that impact this process. Drug discovery has evolved from what was, several decades ago, essentially random screening of natural products, into a scientific process that not only includes the rational and combinatorial design of large numbers of synthetic molecules as potential bioactive agents, such as ligands, agonists, antagonists, and inhibitors, but also the identification, and mechanistic and structural characterization of their biological targets, which may be polypeptides, proteins, or nucleic acids. These key areas of drug design and structural biology are of tremendous importance to the understanding and treatment of disease. However, significant hurdles need to be overcome when trying to identify or develop high affinity ligands for a particular biological target. These include the difficulty surrounding the task of elucidating the structure of targets and targets to which other molecules may be bound or associated, the large numbers of compounds that need to be screened in order to generate new leads or to optimize existing leads, the need to dissect structural similarities and dissimilarities between these large numbers of compounds, correlating structural features to activity and binding affinity, and the fact that small structural changes can lead to large effects on biological activities of compounds.

Traditionally, drug discovery and optimization have involved the expensive and time-consuming, and therefore slow, process of synthesis and evaluation of single compounds bearing incremental structural changes. When using natural products, the individual components of extracts had to be painstakingly separated into pure constituent compounds prior to biological evaluation. Further, all compounds had to be carefully analyzed and characterized prior to in vitro screening. These screens typically included evaluation of candidate compounds for binding affinity to their target, competition for the ligand binding site, or efficacy at the target as determined via inhibition, cell proliferation, activation or antagonism end points. Considering all these facets of drug design and screening that slow the process of drug discovery, a number of approaches to alleviate or remedy these matters, have been implemented by those involved in discovery efforts.

One way in which the drug discovery process is being accelerated is by the generation of large collections, libraries, or arrays of compounds. The strategy of discovery has moved from selection of drug leads from among compounds that are individually synthesized and tested to the screening of large collections of compounds. These collections may be from natural sources (Sternberg et al., Proc. Natl. Acad. Sci. USA, 1995, 92, 1609-1613) or generated by synthetic methods such as combinatorial chemistry (Ecker and Crooke, Bio/Technology, 1995, 13, 351-360 and U.S. Pat. No. 5,571,902, incorporated herein by reference). These collections of compounds may be generated as libraries of individual, well-characterized compounds synthesized, e.g. via high throughput, parallel synthesis or as a mixture or a pool of up to several hundred or even several thousand molecules synthesized by split-mix or other combinatorial methods. Screening of such combinatorial libraries has usually involved a binding assay to determine the extent of ligand-receptor interaction (Chu et al., J. Am. Chem. Soc., 1996, 118, 7827-35). Often the ligand or the target receptor is immobilized onto a surface such as a polymer bead or plate. Following detection of a binding event, the ligand is released and identified. However, solid phase screening assays can be rendered difficult by non-specific interactions.

Whether screening of combinatorial libraries is performed via solid-phase, solution methods or otherwise, it can be a challenge to identify those components of the library that bind to the target in a rapid and effective manner and which, hence, are of greatest interest. This is a process that needs to be improved to achieve ease and effectiveness in combinatorial and other drug discovery processes. Several approaches to facilitating the understanding of the structure of biopolymeric and other therapeutic targets have also been developed so as to accelerate the process of drug discovery and development. These include the sequencing of proteins and nucleic acids (Smith, in Protein Sequencing Protocols, Humana Press, Totowa, N.J., 1997; Findlay and Geisow, in Protein Sequencing: A Practical Approach, IRL Press, Oxford, 1989; Brown, in DNA Sequencing, IRL Oxford University Press, Oxford, 1994; Adams, Fields and Venter, in Automated DNA Sequencing and Analysis, Academic Press, San Diego, 1994). These also include elucidating the secondary and tertiary structures of such biopolymers via NMR (Jefson, Ann. Rep. in Med. Chem., 1988, 23, 275; Erikson et al., Ann. Rep. in Med. Chem., 1992, 27, 271-289), X-ray crystallography (Erikson et al., Ann. Rep. in Med. Chem., 1992, 27, 271-289) and the use of computer algorithms to attempt the prediction of protein folding (Copeland, in Methods of Protein Analysis: A Practical Guide to Laboratory Protocols, Chapman and Hall, New York, 1994; Creighton, in Protein Folding, W. H. Freeman and Co., 1992).

Experiments such as ELISA (Kemeny and Challacombe, in ELISA and other Solid Phase Immunoassays: Theoretical and Practical Aspects; Wiley, New York, 1988) and radioligand binding assays (Berson et al., Clin. Chim. Acta, 1968, 22, 51-60; Chard, in “An Introduction to Radioimmunoassay and Related Techniques,” Elsevier press, Amsterdam/New York, 1982), the use of surface-plasmon resonance (Karlsson, Michaelsson and Mattson, J. Immunol. Methods, 1991, 145, 229; Jonsson et al., Biotechniques, 1991, 11, 620), and scintillation proximity assays (Udenfriend et al., Anal. Biochem., 1987, 161, 494-500) are being used to understand the nature of the receptor-ligand interaction.

All of the foregoing paradigms and techniques are now available to persons of ordinary skill in the art and their understanding and mastery is assumed herein.

Likewise, advances have occurred in the chemical synthesis of compounds for high-throughput biological screening. Combinatorial chemistry, computational chemistry, and the synthesis of large collections of mixtures of compounds or of individual compounds have all facilitated the rapid synthesis of large numbers of compounds for in vitro screening. Despite these advances, the process of drug discovery and optimization entails a sequence of difficult steps. This process can also be an expensive one because of the costs involved at each stage and the need to screen large numbers of individual compounds. Moreover, the structural features of target receptors can be elusive.

One step in the identification of bioactive compounds involves the determination of binding affinity of test compounds for a desired biopolymeric or other receptor, such as a specific protein or nucleic acid or combination thereof. For combinatorial chemistry, with its ability to synthesize, or isolate from natural sources, large numbers of compounds for in vitro biological screening, this challenge is magnified. Since combinatorial chemistry generates large numbers of compounds or natural products, often isolated as mixtures, there is a need for methods which allow rapid determination of those members of the library or mixture that are most active or which bind with the highest affinity to a receptor target.

From a related perspective, there are available to the drug discovery scientist a number of tools and techniques for the structural elucidation of biologically interesting targets, for the determination of the strength and stoichiometry of target-ligand interactions, and for the determination of active components of combinatorial mixtures.

Techniques and instrumentation are available for the sequencing of biological targets such as proteins and nucleic acids (e.g. Smith, in Protein Sequencing Protocols, 1997 and Findlay and Geisow, in Protein Sequencing: A Practical Approach, 1989) cited previously. While these techniques are useful, there are some classes and structures of biopolymeric target that are not susceptible to such sequencing efforts, and, in any event, greater convenience and economy have been sought. Another drawback of present sequencing techniques is their inability to reveal anything more than the primary structure, or sequence, of the target.

While X-ray crystallography is a very powerful technique that can allow for the determination of some secondary and tertiary structure of biopolymeric targets (Erikson et al., Ann. Rep. in Med. Chem., 1992, 27, 271-289), this technique can be an expensive procedure and very difficult to accomplish. Crystallization of biopolymers is extremely challenging, difficult to perform at adequate resolution, and is often considered to be as much an art as a science. Further confounding the utility of X-ray crystal structures in the drug discovery process is the inability of crystallography to reveal insights into the solution-phase, and therefore the biologically relevant, structures of the targets of interest. Some analysis of the nature and strength of interaction between a ligand (agonist, antagonist, or inhibitor) and its target can be performed by ELISA (Kemeny and Challacombe, in ELISA and other Solid Phase Immunoassays: 1988), radioligand binding assays (Berson et al., Clin. 1968, Chard, in “An Introduction to Radioimmunoassay and Related Techniques,” 1982), surface-plasmon resonance (Karlsson et al., 1991, Jonsson et al., Biotechniques, 1991), or scintillation proximity assays (Udenfriend et al., Anal. Biochem., 1987), all cited previously. The radioligand binding assays are typically useful only when assessing the competitive binding of the unknown at the biding site for that of the radioligand and also require the use of radioactivity. The surface-plasmon resonance technique is more straightforward to use, but is also quite costly. Conventional biochemical assays of binding kinetics, and dissociation and association constants are also helpful in elucidating the nature of the target-ligand interactions.

When screening combinatorial mixtures of compounds, the drug discovery scientist will conventionally identify an active pool, deconvolute it into its individual members via resynthesis, and identify the active members via analysis of the discrete compounds. Current techniques and protocols for the study of combinatorial libraries against a variety of biologically relevant targets have many shortcomings. The tedious nature, high cost, multi-step character, and low sensitivity of many of the above-mentioned screening technologies are shortcomings of the currently available tools. Further, available techniques do not always afford the most relevant structural information—the structure of a target in solution, for example. Instead they provide insights into target structures that may only exist in the solid phase. Also, the need for customized reagents and experiments for specific tasks is a challenge for the practice of current drug discovery and screening technologies. Current methods also fail to provide a convenient solution to the need for deconvolution and identification of active members of libraries without having to perform tedious re-syntheses and re-analyses of discrete members of pools or mixtures.

Therefore, methods for the screening and identification of complex chemical libraries especially combinatorial libraries are greatly needed such that one or more of the structures of both the target and ligand, the site of interaction between the target and ligand, and the strength of the target-ligand interaction can be determined. Further, in order to accelerate drug discovery, new methods of screening combinatorial libraries are needed to provide ways for the direct identification of the bioactive members from a mixture and to allow for the screening of multiple biomolecular targets in a single procedure. Straightforward methods that allow selective and controlled cleavage of biopolymers, while also analyzing the various fragments to provide structural information, would be of significant value to those involved in biochemistry and drug discovery and have long been desired. Also, it is preferred that the methods not be restricted to one type of biomolecular target, but instead be applicable to a variety of targets such as nucleic acids, peptides, proteins and oligosaccharides.

Accordingly, the present invention identifies molecular interaction sites in nucleic acids, especially RNA, particularly vimentin RNA. The present invention also identifies secondary structural elements in vimentin RNA which are highly likely to give rise to significant therapeutic, regulatory, or other interactions with “small” molecules and the like. Identification of tissue-enriched unique structures in vimentin RNA is also contemplated.

### SUMMARY OF THE INVENTION

The present invention is directed to an RNA molecule comprising a joined sequence of at least twenty-four nucleotides but not more than seventy nucleotides and having secondary structure defined by three nucleotides forming a first side of a first double stranded region, two nucleotides forming a first side of an internal loop region, four nucleotides forming a first side of a second double stranded region, four or five nucleotides forming an end loop region, four nucleotides forming a second side of said second double stranded region, four nucleotides forming a second side of said internal loop region, and three nucleotides forming a second side of said first double stranded region.

The present invention is also dircted to a purified and isolated RNA molecule comprising a joined sequence of nucleotides having secondary structure defined by three nucleotides forming a first side of a first double stranded region, two nucleotides forming a first side of an internal loop region, four nucleotides forming a first side of a second double stranded region, four or five nucleotides forming an end loop region, four nucleotides forming a second side of said second double stranded region, four nucleotides forming a second side of said internal loop region, and three nucleotides forming a second side of said first double stranded region.

The present invention is also directed to an in silico RNA comprising a joined sequence of nucleotides having secondary structure defined by three nucleotides forming a first side of a first double stranded region, two nucleotides forming a first side of an internal loop region, four nucleotides forming a first side of a second double stranded region, four or five nucleotides forming an end loop region, four nucleotides forming a second side of said second double stranded region, four nucleotides forming a second side of said internal loop region, and three nucleotides forming a second side of said first double stranded region.

The present invention is also directed to an isolated RNA fragment comprising the consensus sequence 5′-NNNNCNNNNNNNUNNANNNNNNNN-3′ (SEQ ID NO:1) or 5′-NNNNCNNNNNNUNNANNNNNNNN-3′ (SEQ ID NO:2), wherein the sequence has a first double stranded region, an internal loop region, a second double stranded region and an end loop region, wherein each of the double stranded and internal loop regions comprises first and second sides, each of the first sides occurring 5′ to the end loop region in the consensus sequence and each of the second sides occurring 3′ to the end loop region in the consensus sequence, and wherein the first and second sides of the internal loop region are unhybridized.

The present invention is also directed to a computer-readable medium encoded with a data structure comprising a representation of an RNA fragment having at least 60% homology across at least two species of organisms comprising the consensus sequence 5′-NNNNCNNNNNNNUNNANNNNNNNN-3′(SEQ ID NO: 1) or 5′-NNNNCNNNNNNUNNA NNNNNNN-3′ (SEQ ID NO:2) and wherein the sequence has a first double stranded region, an internal loop region, a second double stranded region and an end loop region, wherein each of the double stranded and internal loop regions comprises first and second sides, each of the first sides occurring 5′ to the end loop region in the consensus sequence and each of the second sides occurring 3′ to the end loop region in the consensus sequence.

The present invention is also directed to a purified and isolated RNA fragment that is conserved across at least two species comprising the the consensus sequence 5′-NNNNCNNNNNNNUNNANNNNNNNN-3′(SEQ ID NO:1) or 5′-NNNNCNNNNNNUNNA NNNNNNNN-3′(SEQ ID NO:2).

The present invention is also directed to a purified and isolated RNA fragment comprising the human sequence UUUACAACAUAAUCUAGUUUACAGAAAAAUC (SEQ ID NO:3).

The present invention is also directed to an in silico representation of an RNA fragment comprising the human sequence UUUACAACAUAAUCUAGUUUACAGAAAAAUC (SEQ ID NO:3).

### DESCRIPTION OF PREFERRED EMBODIMENTS

The present invention identifies the physical structures present in a target nucleic acid which are of great importance to an organism in which the nucleic acid is present. Such structures—called “molecular interaction sites”—are capable of interacting with molecular species to modify the nature or effect of the nucleic acid. This may be exploited therapeutically as will be appreciated by persons skilled in the art. Such structures may also be found in the nucleic acid of organisms having great importance in agriculture, pollution control, industrial biochemistry, and otherwise. Accordingly, pesticides, herbicides, fungicides, industrial organisms such as yeast, bacteria, viruses, and the like, and biocatalytic systems may be benefitted hereby.

The nucleic acid molecules disclosed herein can be used to screen potential therapeutic compounds including, but are not limited to, organic or inorganic, small to large molecular weight individual compounds, mixtures and combinatorial libraries of ligands, inhibitors, agonists, antagonists, substrates, and biopolymers, such as peptides, nucleic acids or oligonucleotides. As will be appreciated, the present invention provides for the identification of molecules having the ability to modulate RNA comprising the molecular interaction sites. “Modulation” refers to augmenting or diminishing RNA activity or expression. Novel combinations of procedures provide extraordinary power and versatility to the present methods. While it is preferred in some embodiments to integrate a number of processes developed by the assignee of the present application as will be set forth more fully herein, it should be recognized that other methodologies may be integrated herewith to good effect. Thus, while it is greatly advantageous to determine molecular binding sited on RNAs and other molecules in accordance with the teachings of this invention, the interactions of ligands and libraries of ligands with RNA and other molecules identified as being of interest may greatly benefit from other aspects of this invention. All such combinations are within the spirit of the invention.

While there are a number of ways to characterize binding between molecular interaction sites and ligands, such as for example, organic compounds, preferred methodologies are described in, for example, U.S. Ser. No. 09/076,440 (U.S. Pat. No. 6,221,587), Ser. No. 09/076,405 (U.S. Pat. No. 6,253,168), Ser. Nos. 09/076,447, 09/076,206, 09/076,214, and 09/076,404, each of which was filed on May 12, 1998 and each assigned to the assignee of this invention, each of which is incorporated herein by reference in its entirety.

Molecular interaction sites have been identified in vimentin RNA using the methods described in, for example, U.S. Pat. No. 6,221,587. These molecular interaction sites contain secondary structure, that is, have three-dimensional form capable of undergoing interaction with “small” molecules and otherwise, and are expected to serve as sites for interacting with “small” molecules, oligomers such as oligonucleotides, and other compounds in therapeutic and other applications. The 3′-UTR stemloop structure in vimentin mRNA (GenBank # X56134, which is incorporated herein by reference in its entirety) interacts with a 46 kD protein, which is involved in cancer.

Exemplary secondary structures that may be identified include, but are not limited to, bulges, loops, stems, hairpins, knots, triple interacts, cloverleafs, or helices, or a combination thereof. Alternatively, new secondary structures may be identified.

A molecular interaction site is a region of a nucleic acid which has secondary structure. Preferably, the molecular interaction site is conserved between a plurality of different taxonomic species. The nucleic acid can be either eukaryotic or prokaryotic. The nucleic acid is preferably mRNA, pre-mRNA, tRNA, rRNA, or snRNA. The RNA can be viral, fungal, parasitic, bacterial, or yeast. Preferably, the molecular interaction site is present in a region of an RNA which is highly conserved among a plurality of taxonomic species. In accordance with some preferred embodiments of this invention, it will be appreciated that the biomolecules having a molecular interaction site or sites, especially RNAs, may be derived from a number of sources. Thus, such RNA targets can be identified by any means, rendered into three dimensional representations and employed for the identification of compounds which can interact with them to effect modulation of the RNA.

The present invention is directed to oligonucleotides comprising a molecular interaction site that is present in vimentin RNA and in the RNA of at least one, preferably several, additional organisms. The nucleotide sequence of the oligonucleotide is selected to provide the secondary structure of the molecular interaction sites described above. The nucleotide sequence of the oligonucleotide is preferably the nucleotide sequence of vimentin RNA. Alternatively, the nucleotide sequence is of nucleic acid molecule from a plurality of different taxonomic species which also contain the molecular interaction site. The molecular interaction site serves as a binding site for at least one molecule which, when bound to the molecular interaction site, modulates the expression of the RNA in a selected organism.

The present invention is also directed to oligonucleotides comprising a molecular interaction site that is present in vimentin RNA and in at least one additional prokaryotic or eukaryotic RNA, wherein the molecular interaction site serves as a binding site for at least one molecule which, when bound to the molecular interaction site, modulates the expression of the vimentin and/or prokaryotic RNA. The additional prokaryotic or eukaryotic RNA is selected from all eukaryotic and prokaryotic organisms and cells but is not the same organism as the organism containing the vimentin RNA. Oligonucleotides, and modifications thereof, are well known to those skilled in the art. The oligonucleotides of the invention can be used, for example, as research reagents to detect, for example, naturally occurring molecules which bind the molecular interaction sites. The oligonucleotides of the invention can also be used as decoys to compete with naturally-occurring molecular interaction sites within a cell for research, diagnostic and therapeutic applications. Molecules which bind to the molecular interaction site modulate, either by augmenting or diminishing, the expression of the RNA. The oligonucleotides can also be used in agricultural, industrial and other applications.

The present invention is also directed to compositions, including pharmaceutical compositions, comprising the oligonucleotides described above in combination with a pharmaceutical carrier. A “pharmaceutical carrier” is a pharmaceutically acceptable solvent, diluent, suspending agent or any other pharmacologically inert vehicle for delivering one or more nucleic acids to an animal, and are well known to those skilled in the art. The carrier may be liquid or solid and is selected, with the planned manner of administration in mind, so as to provide for the desired bulk, consistency, etc., when combined with the other components of a pharmaceutical composition. Typical pharmaceutical carriers include, but are not limited to, binding agents (e.g., pregelatinised maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrates (e.g., starch, sodium starch glycolate, etc.); or wetting agents (e.g., sodium lauryl sulphate, etc.).

Computational methods employed for the in silico design and synthesis of combinatorial libraries of small molecules are disclosed in, for example, U.S. Pat. No. 6,253,168, which is incorporated herein by reference in its entirety. Methods for tracking and storing the information generated during the in silico creation of library members into relational databases for later access and use are disclosed in, for example, U.S. Pat. No. 6,253,168. For the purposes of this specification, in silico refers to the creation in a computer memory, i.e., on a silicon or other like chip. Stated otherwise in silico means “virtual.” Methods for the one-pot generation of mixtures of compounds by commencing the library generation using different starting fragments in a one-pot fashion are disclosed in, for example, U.S. Pat. No. 6,253,168.

Docking of the library members (or ligands) entails the in silico binding of the members to desired target molecules. Characterization of interactions between the molecular interaction sites in RNA and ligands are described in, for example, International Publication WO 99/58722, which is incorporated herein by reference in its entirety.

Certain preferred evaluation techniques employing mass spectroscopy are disclosed in U.S. Pat. No. 6,329,146 as well as International Publication No. WO 99/45150, each of which is incorporated herein by reference in its entirety.

The present invention is also directed to nucleic acids comprising a joined sequence of at least twenty-four nucleotides but not more than seventy nucleotides and having secondary structure defined by three nucleotides forming a first side of a first double stranded region, two nucleotides forming a first side of an internal loop region, four nucleotides forming a first side of a second double stranded region, four or five nucleotides forming an end loop region, four nucleotides forming a second side of the second double stranded region, four nucleotides forming a second side of the internal loop region, and three nucleotides forming a second side of the first double stranded region. The nucleic acid can be preferably up to 70 nucleotides, 65 nucleotides, 60 nucleotides, 50 nucleotides, 40 nucleotides or 30 nucleotides.

In preferred embodiments, the two nucleotides forming the first side of the internal loop region are of the sequence NC. In other preferred embodiments, the four nucleotides forming the first side of the second double stranded region are of the sequence NNNN and the four nucleotides forming the second side of the second double stranded region are of the sequence NANN. In other preferred embodiments, the four or five nucleotides forming the end loop region are of the sequence NNNUN or NNUN. Preferably, the nucleic acid comprises a portion of vimentin RNA. More preferably, the nucleic acid comprises a portion of the 3′-UTR of vimentin mRNA.

In other preferred embodiments, the nucleic acid fragment comprise the consensus sequence NNNNCNNNNNNNUNNANNNNNNNN (SEQ ID NO: 1) or NNNNCNNNNNNUN NANNNNNNNN (SEQ ID NO:2) and wherein the sequence has a first double stranded region, an internal loop region, a second double stranded region and an end loop region. In other preferred embodiments, an in silico representation of a nucleic acid fragment that is conserved across at least two species comprises the consensus sequence NNNNCNNNNNNNUNNANNN NNNNN (SEQ ID NO: 1) or NNNNCNNNNNNNUNNANNNNNNNN (SEQ ID NO:2). In other preferred embodiments, a purified and isolated nucleic acid fragment that is conserved across at least two species comprises the sequence NNNNCNNNNNNNUNNANNNNNNNN (SEQ ID NO: 1) or NNNNCNNNNNNUNNANNNNNNNN (SEQ ID NO:2). In other preferred embodiments, a purified and isolated nucleic acid fragment comprises the human sequence UUUACAACAUAAUCUAGUUUACAGAAAAAUC (SEQ ID NO:3). In other preferred embodiments, an in silico representation of a nucleic acid fragment comprises the human sequence UUUACAACAUAAUCUAGUUUACAGAAAAAUC (SEQ ID NO:3).

The present invention is also directed to the purified and isolated nucleic acids described above. In addition, the present invention is also directed to the nucleic acids described above in silico.

The following examples are meant to be exemplary of preferred embodiments of the invention and are not meant to be limiting.

The present invention is also directed to data sets comprising the numerical representations of the three dimensional structures of molecular interaction sites and to the numerical representations of the three dimensional structure of a plurality of organic compounds.

### EXAMPLES

### Example 1

### The Iron Responsive Element (Method A)

1. Selecting RNA Target

To illustrate the strategy for identifying small molecule interaction sites, the iron responsive element (IRE) in the mRNA encoded by the human ferritin gene is identified. The IRE is a typical example of an RNA structural element that is used to control the level of translation of mRNAs associated with iron metabolism. The structure of the IRE was recently determined using NMR spectroscopy. In addition, NMR analysis of IRE structure is described in Gdaniec et al., Biochem., 1998, 37, 1505-1512 and Addess et al., J. Mol. Biol., 1997, 274, 72-83. The IRE is an RNA element of approximately 30 nucleotides that folds into a hairpin structure and binds a specific protein. Because this structure has been so well studied and it known to appear in the mRNA of many species, it serves an excellent example of how Applicants' methodology works.

2. Determining Nucleotide Sequence of the RNA Target

The human mRNA sequence for ferritin is used as the initial mRNA of interest or master sequence. The ferritin protein sequence is also used in the analysis, particularly in the initial steps used to find related sequences. In the case of human ferritin gene, the best input is the full length annotated mRNA and protein sequence obtained from UNIGENE. However, for many genes of interest the same level of detailed information is not available. In these cases, alternative sources of master sequence information is obtained from sources such as, for example, GenBank, TIGR, dbEST division of GenBank or from sequence information obtained from private laboratories. Applicants' methods work using any level of input sequence information, but requires fewer steps with a high quality annotated input sequence.

3. Identifying Similar Sequences

An early step in the process is to use the master sequence (nucleotide or protein) to find and rank related sequences in the database (orthologs and paralogs). Sequence similarity search algorithms are used for this purpose. All sequence similarity algorithms calculate a quantitative measure of similarity for each result compared with the master sequence. An example of a quantitative result is an E-value obtained from the Blast algorithm. The E-values for a blast search of the non-redundant GenBank database using ferritin mRNA as the query sequence illustrates the use of quantitative analysis of sequence similarity searches. The E-value is the probability that a match between a query sequence and a database sequence occurs due to random chance. Therefore, the lower an E-value the more likely that two sequences are truly related. Sequences that meet the cutoff criteria are selected for more detailed comparisons according to a set of rules described below. Since an objective of the sequence similarity search to find distantly related orthologs and paralogs, it is preferable that the cutoff criteria not be too stringent, or the target of the search may be excluded.

4. Identification of Conserved Regions

Identification of conserved regions is performed by pairwise sequence comparisons using Q-Compare in conjunction with CompareOverWins. Conservation of structure between genes with related function from different species is a major indication that can be used to find good drug binding sites. Conserved structure can be identified by using distantly related sequences and piecing together the remnants of conserved sequence combining it with an analysis of potential structure. Sequence comparisons are made between pairs of mRNAs from different species using Q-compare that can identify traces of sequence conservation from even very divergent organisms. Q-compare, in conjunction with CompareOverWins, compares every region of each sequence by sliding one sequence over the other from end to end and measuring the number of matches in a window of a specific size.

When the human mRNA and mouse mRNA sequences for ferritin, which each contain an IRE in the 5′-UTR, are analyzed in this manner, a plot showing the regions of sequence similarity is produced. Pairwise analysis of the human and mouse ferritin mRNA sequences illustrate several important aspects of this type of analysis. Regions of each mRNA that encode the amino acid sequence have the highest degree of similarity, while the untranslated regions are less similar. In both the human and mouse ferritin mRNAs the IREs are located in the extreme 5′ end of each mRNA. This demonstrates an important point—the sequence conservation in the region of the IRE structure does not stand out against the background of sequence similarity between the human and mouse ferritin sequences. In contrast, in the comparison of human and trout or human and chicken ferritin mRNAs, the IREs can be immediately identified. This is because the sequence of the UTRs between human and trout or human and chicken are separated by greater evolutionarily distance than human and mouse, which is logical in view of the evolutionary distance that separates humans from birds and fish compared with other mammals. Comparing the human sequence to that of birds and fish is informative because the natural drift due to evolution has allowed many sequence changes in the UTRs. However, the IRE sequences are more constrained because they form an important structure. Thus, they stand out better and can be more readily identified.

The same principle applies when comparing the trout and chicken ferritin sequences to each other. While both are separated from humans by hundreds of millions of years of evolution, they are also well separated from each other. This illustrates another important tactic used in the present invention—comparison of two non-human RNA sequences can be used to find a regulatory RNA structure without having the actual human sequence. The non-human comparison work can actually direct one skilled in the art where to look to find a human counterpart as a potential drug target.

Evolutionary distances can be used to decide which sequences not to compare as well as which to compare. As with the human and mouse, comparison of trout and salmon are less informative because the species are too close and the IRE does not stand out above the UTR background. Comparison of human and Drosophia ferritin mRNA sequences fail to find the IREs in either species, even though they are present. This is because the sequence of the IREs between humans and Drosophila have diverged even though the structure is conserved. However, if the Drosophila and mosquito ferritin mRNAs are compared, the IREs are identified, again illustrating that the human sequence need not be in hand to identify a regulatory element relevant to drug discovery in humans.

The software used in the present invention makes the decision whether or not to compare sequences pairwise using a lookup table based upon the evolutionary distances between species. The lookup table in the present invention includes all species that have sequences deposited in GenBank. Q-Compare in conjunction with CompareOverWins decides which sequences to compare pairwise.

5. Identification Of Secondary Structure

Sets of sequences that show evidence of conservation in orthologs and paralogs or other related genes are analyzed for the ability to form internal structure. This is accomplished by analyzing each sequence in a matrix where the sequence is plotted 5′ to 3′ on the X axis and its reverse complement is plotted 5′ to 3′ on the Y axis, such as in, for example, self-complementary analysis. Matches that correspond to potential intramolecular base pairs are scored according to a table of values. When the human ferritin IRE sequence is analyzed in this fashion, the diagonals indicate potential self-complementary regions. Each of the 13 IRE sequences described in this example were analyzed in the same fashion. While each of the sequences can form a variety of different structures, the structure most likely to occur is one common to all the sequences. By superimposing the plots of all 13 individual sequences, the potential structure common to all the sequences is deduced.

### Example 2

### The Iron Responsive Element (Method B)

2. Determining Nucleotide Sequence of the RNA Target

The human mRNA sequence for ferritin was used as the initial mRNA of interest or master sequence. The ferritin protein sequence was also used in the analysis, particularly in the initial steps used to find related sequences. In the case of human ferritin gene, the best input is the full length annotated mRNA (gi507251) and protein sequence obtained from UNIGENE. However, for many genes of interest the same level of detailed information is not available. In these cases, alternative sources of master sequence information is obtained from sources such as, for example, Hovergen and GenBank. The present methods work using any level of input sequence information, but requires fewer steps with a high quality annotated input sequence.

3. Identifying Similar Sequences

An alternate, and preferred, approach to finding orthologs is the use of Hovergen database and query tools that have been described in Duret et al., Nuc. Acids Res., 1994, 22, 2360-2365, which is incorporated herein by reference in its entirety. Hovergen was used to identify related sequences (tree classification at the species level classification at the order level). Sequences corresponding to each of these orthologs was saved in GenBank format and grouped together in a single data file. Untranslated regions in both the 5′ and 3′ flanks of the coding region was extracted using SEALS and COWX.

4. Identification of Conserved Regions

The IRE sequences are more constrained because they form an important structure. Thus, they stand out better and can be more readily identified even in closely related sequences. However, for this to work for any gene, the compare algorithm has been rewritten. This new tool, CompareOverWins, allows a dynamic selection of both the range of window sizes, as well the hit threshold. This algorithm needs as its input parsed and separated 5′ and 3′-UTR sequences. Tools available within the Seals genome analysis package described earlier can be used to achieve this.

To identify the IRE using the methods described herein, the compare over windows algorithm was used and the results visualized using AlignHits. In addition to optimizing the thresholding, CompareOverWins also extracts the sequence corresponding to the hits. ClustalW (version 1.74) was used on the extracted sequences to create a locally gapped alignment.

5. Identification Of Secondary Structure

Sets of sequences that show evidence of conservation in orthologs and paralogs or other related genes are analyzed for the ability to form internal structure. This is accomplished by analyzing each sequence in a matrix where the seqeunce is plotted 5′ to 3′ on the X axis and its complement is plotted 5′ to 3′ on the Y axis, such as in, for example, self-complementary analysis. Matches that correspond to potential intramolecular base pairs are scored according to a table of values. When the human ferritin IRE sequence is analyzed in this fashion, the diagonals indicate potential self-complementary regions. Each of the 13 IRE sequences described in this example were analyzed in the same fashion. While each of the sequences can form a variety of different structures, the structure most likely to occur is one common to all the sequences. By superimposing the plots of all 13 individual sequences, the potential structure common to all the sequences is deduced.

The above scheme has been implemented algorithmically into a program called RevComp. RevComp creates a sorted list of all the structures. Representative results can be viewed either as a “dome” ouptut or as a “connect” or “ct” file which can be used in one of many RNA structure viewing programs (RNAStructure, RNAViz, etc.)

### Example 3

### Histone

Histone 3′-UTR represents another classic stem-loop structure that has been studied extensively (EMBO, 1997, 16, 769). At the post-transcriptional level, the stem-loop structure in the 3′ untranslated region of the histone mRNA has been shown to be very important. Son, Saenghwahak Nyusu, 1993, 13, 64-70. The analysis shown below describes the use of this known structure to validate the strategy and methods described herein.

Phylogenetic tree outputs for all Histone orthologs in Hovergen database was obtained. Each of these orthologs was saved in GenBank format and grouped together in a single data file. Untranslated regions in both the 5′ and 3′ flanks of the coding regions were extracted and compared using SEALS and COWX as described earlier.

Following extraction and comparison by SEALS and COWX, Align Hits was used to determine potentially interesting regions. The sequences corresponding to the region of interest was extracted from all species for alignment with CLUSTAL W (1.74). Following extraction of sequence information from Align Hits, CLUSTAL W (1.74) was used to provide multiple sequence alignment shown. Each of the putative hit sequences was analyzed for the ability to form internal structure. This was accomplished by analyzing each sequence in a matrix where the sequence was plotted 5′ to 3′ on the X axis and its complement is plotted 5′ to 3′ on the Y axis. Base-pairs along the diagonals indicate potential self-complementary regions that can form secondary structures. A representative sequence alignment in a dome format can show potential stem formation between the base pairs. Following conversion of the dome format file to a ct file, RNA Structure 3.21 is used to visualize the structure.

### Example 4

### Vimentin

Vimentin is an intermediate filament protein whose 3′-UTR is highly conserved between species. Previous studies by Zehner et al., (Nuc. Acids Res., 1997, 25, 3362-3370) has shown that a proposed a complex stem-loop structure contained within this region may be important for vimentin mRNA functions such as mRNA localization. The same region was identified using the present analysis, thus, validating the present approach. In addition, based on the analyses described herein, a second stem-loop structure that occurs downstream of the previously proposed structure that may have a role in regulating vimentin fuction as well has been identified.

A representative phylogenetic tree output for all Vimentin orthologs in Hovergen database was obtained. Each of these orthologs was saved in GenBank format and grouped together in a single data file. Untranslated regions in both the 5′ and 3′ flanks of the coding regions were extracted and compared using SEALS and COWX as described earlier.

Following extraction and comparison by SEALS and COWX, Align Hits was used to determine potentially interesting regions. Two such regions appeared, and were used for subsequent analyses. Following extraction of sequence information from Align Hits for the first region, CLUSTAL W was used to provide multiple sequence alignment. Potential stem formation between base pairs was given above the sequence alignment in a dome format. Following conversion of the dome format file to a ct file, RNA Structure 3.21 was used to visualize the structure. This structure is very similar to the one proposed by Zehner et al. Zehner et al. presented a detailed chemical analysis of their proposed structure for the minimal binding domain in the 3′-UTR of Vimentin. This analysis included cleavage with single-strand-specific (ChS or T1) or double-strand-specific (V1) nucleases as well as after exposure to lead acetate.

Following extraction of sequence information from Align Hits for the second region, CLUSTAL W was used to provide multiple sequence alignment. The potential stem formation between base pairs in the second region was given above the sequence alignment in a dome format. Following conversion of the dome format file to a ct file, RNA Structure 3.21 was used to visualize the structure for the second region.

### Example 5

### Transferrin Receptor

Similar to regulation of ferritin Examples 1 and 2), another known function of the IRE is in the regulation of transferrin receptor. Five IREs have been identified in the 3′-UTRs of known transferring receptor mRNAs. Kuhn et al., EMBO J., 1987, 6, 1287-93 and Casey et al., Science, 1988, 240, 924-928, each of which is incorporated herein by reference in its entirety. All 5 IREs have been shown to interact with iron regulatory proteins (IRP) independently. The present techniques were applied to identify these conserved elements in transferrin receptors.

A representative phylogenetic tree output for all Transferrin receptor orthologs in Hovergen database was obtained. Each of these orthologs was saved in GenBank format and grouped together in a single data file. Untranslated regions in both the 5′ and 3′ flanks of the coding region were extracted and compared using SEALS and COWX as described earlier.

Following extraction and comparison by SEALS and COWX, Align Hits was used to determine potentially interesting regions. The first region, between base pairs 920 to 990, in the 3′-UTR of transferrin receptor was extracted from all species for alignment with CLUSTAL W (1.74).

Following extraction of sequence information from Align Hits for the first region, CLUSTAL W (1.74) was used to provide multiple sequence alignment. A representative potential stem formation between base pairs was given above the sequence alignment in a dome format. Following conversion of the dome format file to a ct file, RNA Structure 3.21 was used to visualize the structure. The second region, between base pairs 990 to 1050, in the 3 prime UTR of transferrin receptor was extracted from all species for alignment with CLUSTAL W (1.74).

Following extraction of sequence information from Align Hits for the second region, CLUSTAL W (1.74) was used to provide multiple sequence alignment. Potential stem formation between base pairs was given above the sequence alignment in a dome format. Following conversion of the dome format file to a ct file, RNA Structure 3.21 was used to visualize the structure. Following extraction and comparison by SEALS and COWX, Align Hits was used to determine potentially interesting regions. The third region, between base pairs 1372 to 1423, in the 3′-UTR of transferrin receptor was extracted from all species for alignment with CLUSTAL W (1.74).

Following extraction of sequence information from Align Hits for the third region, CLUSTAL W (1.Ex.34) was used to provide multiple sequence alignment. Potential stem formation between base pairs was given above the sequence alignment in a dome format. Following conversion of the dome format file to a ct file, RNA Structure 3.21 was used to visualize the structure. Following extraction and comparison by SEALS and COWX, Align Hits was used to determine potentially interesting regions. The fourth region, between base pairs 1439 to 1479, in the 3′-UTR of transferrin receptor was extracted from all species for alignment with CLUSTAL W (1.74).

Following extraction of sequence information from Align Hits for the fourth region, CLUSTAL W (1.Ex.34) was used to provide multiple sequence alignment. Potential stem formation between base pairs was given above the sequence alignment in a dome format. Following conversion of the dome format file to a ct file, RNA Structure 3.21 was used to visualize the structure. Following extraction and comparison by SEALS and COWX, Align Hits was used to determine potentially interesting regions. The fifth region, between base pairs 1479 to 1542, in the 3′-UTR of transferrin receptor was extracted from all species for alignment with CLUSTAL W (1.74).

Following extraction of sequence information from Align Hits for the fifth region, CLUSTAL W (1.Ex.34) was used to provide multiple sequence alignment. Potential stem formation between base pairs was given above the sequence alignment in a dome format. Following conversion of the dome format file to a ct file, RNA Structure 3.21 was used to visualize the structure.

### Example 6

### Ornithine Decarboxylase

Orinithine decarboxylase (ODC) is the first enzyme in the polyamine biosynthetic pathway. Studies have shown existence of translational regulatory elements both in the 5′ and 3′ untranslated regions (Grens et al., J. Biol. Chem., 1990, 265, 11810). Secondary structures have been proposed to exist in both these regions, though there is no conclusive evidence for it. The methods described herein identified two structures in the 3′-UTR, as shown below. The presence of one of these structures was verified using mass spectrometry probing (Griffey, et al., Proc. SPIE-Int. Soc. Opt. Eng., 2985 (Ultrasensitive Biochemical Diagnostics II): 82-86, which is incorporated herein by reference in its entirety). Two representative sequences that showed slight variation in their lengths were made into RNA and subjected to MS structure probing. Results confirm the presence of a stem-loop structure. Accordingly, identification of a novel secondary structure can be identified from the methods described herein, and such existence has been independently verified by structure probing.

Phylogenetic tree outputs for all Ornithine Decarboxylase orthologs in Hovergen database were obtained. Each of these orthologs was saved in GenBank format and grouped together in a single data file. Untranslated regions in both the 5′ and 3′ flanks of the coding region were extracted and compared using SEALS and COWX as described earlier.

Following extraction and comparison by SEALS and COWX, Align Hits was used to determine potentially interesting regions. Two such regions appeared, and were used for subsequent analyses. Following extraction of sequence information from the first region, CLUSTAL W (1.74) was used to provide multiple sequence alignment shown. Each of the putative hit sequences was analyzed for the ability to form internal structure in a reverse complement matrix. This was accomplished by analyzing each sequence in a matrix where the sequence is plotted 5′ to 3′ on the X axis and its complement is plotted 5′ to 3′ on the Y axis. Base-pairs along the diagonals indicate potential self-complementary regions that can form secondary structures. Domes view of the potential stem formation between base pairs in region 1 is given above the sequence alignment was determined using RevComp. RNA Structure 3.2 was used to visualize the structure.

Mass spectrometry analyses techniques were used to probe for structure. The cluster alignment of the first region of ornithine decarboxylase 3′-UTR showed presence of gaps/inserts in the multiple alignment. Two representative RNAs (gi404561 and gi35135) from the alignments were used for this experiment. Analysis of the pattern of induced fragmentation showed a very strong likelihood for base-paring along the top half of the stem-loop structure. This corresponds to bases 11-14 and 20-23 in 404561 or bases 8-11 and 18-21 in 35135. Bulged bases (G9 in 404561 or U22 in 35135) also showed characteristic fragmentation pattern. The bottom-half of the structure appeared to be less stable, and showed some fragmentation where our analyses had predicted base-paring. This was particularly true in the sequence 35135. This region, however, has several contiguous A-U or G-U base-pairs which tend to be less stable, and therefore have a higher probability of fragmentation.

Following extraction of sequence information from Align Hits for the second region, CLUSTAL W was used to provide multiple sequence alignment. Potential stem formation between base pairs in the second region was given above the sequence alignment in a dome format. Following conversion of the dome format file to a ct file, RNA Structure 3.21 was used to visualize the structure for the second region.

### Example 7

### Interleukin-2 (IL-2)

A representative phylogenetic tree output for all IL-2 orthologs in Hovergen database was obtained. Each of these orthologs was saved in GenBank format and grouped together in a single data file. Untranslated regions in both the 5′ and 3′ flanks of the coding region were extracted and compared using SEALS and COWX as described earlier.

Following extraction and comparison by SEALS and COWX, Align Hits was used to determine potentially interesting regions in the 3′-UTR region. Two such regions appear, and were used for subsequent analyses. Following extraction of sequence information from Align Hits for the first region, CLUSTAL W (1.74) was used to provide multiple sequence alignment. Domes view of the potential stem formation between base pairs in the first region was given above the sequence alignment using RevComp. RNA Structure 3.2 was used to visualize the structure. Following extraction of sequence information from Align Hits for the second region, CLUSTAL W (1.74) was used to provide multiple sequence alignment. Potential stem formation between base pairs in the second region was given above the sequence alignment in a dome format. Following conversion of the dome format file to a ct file, RNA Structure 3.21 was used to visualize the structure for the second region.

In addition to the two regions described above, a third region, downstream of, and partially overlapping the second region, was identified using an alternate reference sequence (3087784.fa). Following extraction of sequence information from Align Hits for this region, CLUSTAL W (1.74) was used to provide multiple sequence alignment. Potential stem formation between base pairs in the third region was shown above the sequence alignment in a dome format. Following conversion of the dome format file to a ct file, RNA Structure 3.21 was used to visualize the structure for the third region.

### Example 8

### Interleukin-4 (IL-4)

Representative phylogenetic tree output for all IL-4 orthologs in Hovergen database was obtained. Each of these orthologs was saved in GenBank format and grouped together in a single data file. Untranslated regions in both the 5′ and 3′ flanks of the coding region were extracted and compared using SEALS and COWX as described earlier.

Following extraction and comparison by SEALS and COWX, Align Hits was used to determine potentially interesting regions in the 5′-UTR region. Following extraction of sequence information from Align Hits for the above region, CLUSTAL W (1.74) was used to provide multiple sequence alignment. Domes view of the potential stem formation between base pairs in the region was given above the sequence alignment using RevComp. RNA Structure 3.2 was used to visualize the structure.

Align Hits was used to view hits in the 3′-UTR region of IL-4. Following extraction of sequence information from Align Hits for the 3′-UTR region, CLUSTAL W (1.74) was used to provide multiple sequence alignment. Potential stem formation between base pairs in the second region was given above the sequence alignment in a dome format. Following conversion of the dome format file to a ct file, RNA Structure 3.21 was used to visualize the structure for the second region.

### Example 9

### General Procedure for Automated Synthesis of Library Plates

ArgoGel-OH™ (360 mg, loading 0.43 mmole/g) was suspended in ˜16 mL solution of 3:1 CH₂Cl₂/DMF. The suspension was distributed equally among 12 wells of a 96 well polypropylene synthesis plate (30 mg per well). The solvent was drained and the resin dried overnight in vacuo over P₂O₅. All solid reagents were dried in vacuo overnight over P₂O₅ prior to use. For method 1, the Mitsunobu reagent 1 was dried, then dissolved in anhydrous CH₂Cl₂ to a concentration of 0.15M. FMOC-Amino Acids (Novabiochem, Bachem Calif.) were dissolved to a concentration of 0.30 M in a solution of 2:1 anhydrous CH₂Cl₂/DMF for method 1, and to a concentration of 0.22 M in DMF containing 0.44 M collidine for synthesis for method 2. Sulfonyl chlorides were dissolved to a concentration of 0.2M in Pyridine. Pyridine proved to be an acceptable solvent for most sulfonyl chlorides, but when solubility was limited, cosolvents such as MeCN, DMSO, CH₂Cl₂, DMF, and NMP (up to 50%) have been employed. FMOC protection were removed with a solution of 10% piperidine in anhydrous DMF prepared and used the day of synthesis. Low water wash solvents were employed to ensure maximum coupling efficiency of the initial amino-acid to the resin. Prior to loading reagents, moisture sensitive reagent lines were purged with argon for 20 minutes. Reagents were dissolved to appropriate concentrations and installed on the synthesizer. Large bottles (containing 8 delivery lines) were used for wash solvents and the delivery of activator. Small septa bottles containing the amino acids and sulfonyl chlorides allow anhydrous preparation and efficient installation of multiple reagents by using needles to pressurize the bottle, and as a delivery path. After all reagents were installed, the lines were primed with reagent, flow rates measured, then entered into the reagent table (.tab file) and the dry resin loaded plate removed from vacuum and installed in the machine for subsequent synthesis. After cleavage from support and centrifugal evaporation of solvent, the products were dissolved in MeOH/CH₂Cl₂ mixtures, then assayed for purity by TLC (typically 10% MeOH/CH₂Cl₂) on silica gel using both UV and I2 visualization, and for product identity by electrospray mass spectroscopy (negative mode). Selected samples were dissolved in DMSO-d₆ and examined by ¹H NMR.

### Example 10

### General Hydroxamic Acid Synthesis Method 1

The commercial ArgoGel-OH™ resin (10 μmole) was washed with CH₂Cl₂ (6×), then treated with the appropriate FMOC-amino acid (3 eq.) and 1 (3 eq.). After 30 min, the wells were drained, and the process repeated to give a total of 4 treatments (12 eq.). The resin was washed with CH₂Cl₂ (6×), DMF (4×), and the FMOC removed with 10% piperidine in DMF (4×). The washes were collected, diluted appropriately, and the amount of FMOC chromophore released quantitated by UV (∈7800 L*mol⁻¹*cm⁻¹, λ=301 nm). This value was used to calculate the yield of the final products. The resin was then washed with DMF (4×), then CH₂Cl₂ (6×), and treated with the appropriate sulfonyl chloride (4×6 eq. for 15 min.) in pyridine, and washed with CH₂Cl₂ (6×), DMF (6×), and CH₂Cl₂ (10×). At this point the resin could be treated with 90:5:5 TFA/H₂O/Et₃SiH for 4 h, then subjected to the above washing procedure to remove any side chain protection on the molecules if necessary. The plates were then removed from the instrument, and individual wells treated with 4 M hydroxylamine (50% aqueous) in 1,4-dioxane for 24 h. The filtrate was collected into a deep well 96 well plate, the samples frozen, then lyophilized to provide the desired hydroxamic acids. Addition of fresh 1,4-dioxane and repetition of the lyophilization process twice gave compounds free of any residual hydroxylamine (by ¹H NMR of selected products).

### Example 11

### General Hydroxamic Acid Synthesis Method 2

Resin 6 was prepared from ArgoGel-Wang-OH™ resin according to published procedures and this resin (10 μmole) was washed with DMF (6×), CH₂Cl₂ (6×), then treated with the appropriate FMOC-amino acid (3 eq.) in DMF +collidine (6 eq.) and HATU (3 eq.). After 30 min, the wells were drained, and the process repeated to give a total of 4 treatments (12 eq.). The resin was washed with CH₂Cl₂ (6×), DMF (4×), and the FMOC removed with 10% piperidine in DMF (4×). The washes were collected, diluted appropriately, and the amount of FMOC chromophore released quantitated by UV (∈7800 L*mol⁻¹*cm⁻¹, λ=301 nm). This value was used to calculate the yield of the final products. The resin was washed with DMF (4×), then CH₂Cl₂ (6×), and treated with the appropriate sulfonyl chloride (4×6 eq. for 15 min.) in pyridine, and washed with CH₂Cl₂ (6×), DMF (8×), DMSO (8×), and CH₂Cl₂ (10×). The plates were then removed from the instrument, and individual wells treated with 90:5:5 TFA/Et₃SiH/H₂O for 4 h. The filtrate was collected into a deep well 96 well plate, the resin washed (3×) with TFA, and the samples concentrated in a centrifugal vacuum concentrator. Addition of fresh 1,4-dioxane or isopropanol and repetition of the concentration process twice, followed by drying in vacuo overnight gave the desired hydroxamic acids.

The methods of both Examples 2 and 3 were utilized to produce a library of compounds resulting from the combination of FMOC-amino acids and sulfonyl chlorides shown in Table 1.

TABLE 1
Reagents Used to Prepare Hydroxamic Acids 5
by Automated Synthesisa

| FMOC-Amino Acid Usedb | | Sulfonyl Chloride Usedc | |
|---|---|---|---|
| a | d-Vald | i | 1-napthalene |
| b | d-Ile | ii | 2-napthalene |
| c | d-Leu | iii | 2-thiophene |
| d | d-Ala | iv | 2-mesitylene |
| e | d-cyclo-hexyl-Ala | v | 3-nitrobenzene |
| f | d-norvaline | vi | 4-bromobenzene |
| g | d-norleucine | vii | 4-chlorobenzene |
| h | d-alloiso-leucine | viii | 4-iodobenzene |
| i | d-α-t-Butylglycinee | ix | 4-nitrobenzene |
| j | d-Met | x | 4-methoxybenzened |
| k | d-Phenyl-glycine | xi | 4-t-Butylbenzene |
| l | d-Phe | xii | trifluoromethaned |
| m | d-4-Chloro-Phe | xiii | -toluene |
| n | 3-(2-napthyl)-d-Ala | xiv | 3-(trifluoromethyl)benzene |
| o | 3-(3-pyridyl)-d-Ala | xv | 4-(trifluromethoxy)benzene |
| p | -(2-thienyl)-d-Ala | xvi | 4-(methylsulfonyl)benzene |
| q | d-Tyr(tBu)d | xvii | 4-(benzenesulfonyl)thiophene-2- |
| r | d-Trp | xviii | 4-ethylbenzene |
| s | d-Cys(tBu) | xix | 4-cyanobenzene |
| t | S-Bn-d-penicillamine | xx | 4-methoxy-2,3,6-trimethylbenzene |
| u | glycine | xxi | benzo-2,1,3-thiadiazole-4- |
| v | aminoisobutyric acid | xxii | 1-Methylimidazole-4- |
| w | d-Thr(tBu)e | xxiii | 5-chloro-3- |
| | | | methylbenzo[B]thiophene-2-d |
| x | d-Ser(tBu) | xxiv | benzofurazan-4- |
| y | d-His(Trt)d | xxv | 3,5-dichlorobenzene |
| z | d-Pro | xxvi | 3,4-dimethoxybenzene |
| aa | d-Tic | xxvii | 4-(n-butoxy)benzene |
| bb | d-Lys(BOC) | xxviii | 2,4-dichlorobenzene |
| cc | d-Asp(OtBu) | xxix | 4-trifluoromethylbenzene |
| dd | d-Glu(OtBu) | xxx | 2,5-dimethoxybenzene |
| ee | l-Val | xxxi | 3,4-dichlorobenzened |
| ff | l-Ala | xxxii | 4-n-propylbenzened |
| gg | l-Phed | xxxiii | 4-isopropylbenzened |
| hh | d-Asn(Trt)e | xxxiiv | 2,5-dichlorothiophene-3- |
| ii | d-Gln(Trt)e | xxxv | 2-[1-methyl-5- |
| | | | (trifluoromethyl)pyrazol-3- |
| | | | yl]thiophene-5- |
| jj | d-Arg(Pmc)d | xxxvi | 2-[3-(trifluoromethyl)pyrid-2-yl |
| | | | sulfonyl]thiophene-5- |

^{a}All possible combinations of reagents shown were utilized to attempt the preparation of 1296 hydroxamic acids according to method 2.
^{b}Standard abbreviations used for FMOC-amino acids. All amino acids used were obtained from Novabiochem, Bachem, or Synthetech.
^{c}Truncated chemical names are given in the table. Appending “sulfonyl chloride” to the prefix listed gives the appropriate name. All sulfonyl chlorides used were obtained from Aldrich, Lancaster, or Maybridge.
^{d}Also prepared via method 1.
^{e}Failed in method 1.

### Example 12

### Representative Parallel Array Synthesizer Input Files

The software inputs accept tab delimited text files from any text editor. Examples for the synthesis of hydroxamic acids are shown in Table 2 (.cmd file), Table 3 (.seq file), and Table 4 (.tab file). Only several wells worth of synthesis are shown for brevity. For an entire plate to be prepared, only additional sulfonyl chlorides and additional amino acids need to be added to the .tab file, and additional combinations of the two need to be added to the .seq file such that it contains 96 lines, with each line corresponding to a unique compound prepared.

The identity and purity of the compounds was determined by electrospray mass spectroscopy (negative mode) and thin layer chromatography on silica employing MeOH/CH₂Cl₂ solvent mixtures (TLC). The synthesis products in approximately every third well were assayed by TLC and electrospray mass spectroscopy, and the desired compounds were generally present with purities of 60 to 90% when using either of the synthesis methods described above.

TABLE 2
Example .cmd file (general synthesis procedure) which executes the
synthesis. The cleavage from support with hydroxylamine is performed
separately.

INITIAL_WASH

BEGIN

Repeat 6

Add CH₂Cl₂ 300
Drain 20

End_Repeat

END

COUPLE_AMINO_ACID

BEGIN

Repeat 4

Add <SEQ> 100 + <ACT1> 200
Wait 1800
Drain 20

End_Repeat
Repeat 6

Add CH₂Cl₂ 300
Drain 20

End_Repeat
Repeat 4

Add DMF 300
Drain 20

End_Repeat

END

REMOVE_FMOC

BEGIN

Load_Tray
Repeat 4

Add PIPERIDINE_DMF 300
Wait 250
Drain 20

End_Repeat
Remove_Tray
Repeat 4

Add DMF 300
Drain 20

End_Repeat
Repeat 6

Add CH₂Cl₂ 300
Drain 20

End_Repeat

END

SULFONYLATE_AMINO_ACID

BEGIN

Next_Sequence
Repeat 4

Add <SEQ> 300
Wait 900
Drain 20

End_Repeat
Repeat 6

Add CH₂Cl₂ 300
Drain 20

End_Repeat

END

FINAL_WASH

BEGIN

Repeat 6

Add DMF 300
Drain 20

End_Repeat
Repeat 8

Add CH₂Cl₂ 300
Drain 20

End_Repeat
Repeat 2

Add CH₂Cl₂ 300
Drain 60

End_Repeat

END

TABLE 3
Example .seq File (list of compounds to make)

| | | | | |
|---|---|---|---|---|
| 1 | A1 | 10 | FMOC_D_ALA | 4_MEO_BENZENE_SO₂CL |
| 2 | A2 | 10 | FMOC_D_VAL | 2_NAPTHYLENE_SO₂CL |
| 3 | A3 | 10 | FMOC_D_PHE | 3_CF_{3—}BENZENE_SO₂CL |
| 4 | A4 | 10 | FMOC_D_NAL | 4_CL_BENZENE_SO₂CL |
| 5 | A5 | 10 | FMOC_D_{—} | 4_MEO_BENZENE_SO₂CL |
| | | | SER(OTBU) | |
| 6 | A6 | 10 | FMOC_D_{—} | 2_NAPTHYLENE_SO₂CL |
| | | | ARG_PMC | |
| 7 | A7 | 10 | FMOC_D_ALA | 3_CF_{3—}BENZENE_SO₂CL |
| 8 | A8 | 10 | FMOC_D_VAL | 4_CL_BENZENE_SO₂CL |
| 9 | A9 | 10 | FMOC_D_PHE | 4_MEO_BENZENE_SO₂CL |
| 10 | A10 | 10 | FMOC_D_NAL | 2_NAPTHYLENE_SO₂CL |
| 11 | A11 | 10 | FMOC_D_{—} | 3_CF_{3—}BENZENE_SO₂CL |
| | | | SER(OTBU) | |
| 12 | A12 | 10 | FMOC_D_{—} | 4_CL_BENZENE_SO₂CL |
| | | | ARG_PMC | |

TABLE 4
Example .tab (list of reagents to use)

AMINO_ACIDS
BEGIN

| | | | |
|---|---|---|---|
| 1 | FMOC_D_ALA | 265 | 0.30 |
| 2 | FMOC_D_VAL | 265 | 0.30 |
| 3 | FMOC_D_PHE | 265 | 0.30 |
| 4 | FMOC_D_NAL | 265 | 0.30 |
| 5 | FMOC_D_SER(OTBU) | 265 | 0.30 |
| 6 | FMOC_D_ARG_PMC | 265 | 0.30 |

END
SOLVENTS
BEGIN

| | | | |
|---|---|---|---|
| 67 | CH₂CL₂ | 330 | 1 |
| 66 | DMF | 240 | 1 |

END
SULFONYLCHLORIDES
BEGIN

| | | | |
|---|---|---|---|
| 9 | 4_MEO_BENZENE_SO₂CL | 220 | 0.20 |
| 10 | 2_NAPTHYLENE_SO₂CL | 220 | 0.20 |
| 11 | 3_CF₃_BENZENE_SO₂CL | 220 | 0.20 |
| 12 | 4_CL_BENZENE_SO₂CL | 220 | 0.20 |

END
DEBLOCK
BEGIN

| | | | |
|---|---|---|---|
| 68 | PIPERIDINE_DMF | 230 | 1 |

END
ACTIVATORS
BEGIN

| | | | | |
|---|---|---|---|---|
| 69 | BETAINE | 300 | 0.15 | Activates |
| | | | | AMINO_ACIDS |

END

### Example 13

### Manual Solution Synthesis of Active Compounds

Methyl (2R)-2-amino-3-(2-naphthyl)Propanoate

To a suspension of D-napthylalanine hydrochloride (2.15 g, 10 mmole, Bachem Calif.) in MeOH (17 mL) was added TMS-Cl (2.8 mL, 22 mmole) dropwise with stirring. The mixture was allowed to stir overnight, and the resulting solution concentrated in vacuo, then dried over KOH to afford 2.65 g (100%) of methyl (2R)-2-amino-3-(2-naphthyl)propanoate, which was >95% pure by ¹H NMR, and used without further purification: R_{f} 0.63 (4:1:1 n-BuOH/AcOH/H₂O); ¹H NMR (DMSO-d₆) δ 8.76 (bs, 3H), 8.00-7.30 (m, 7H), 4.39 (t, 1H), 3.69 (s, 3H), 3.66 (m, 2H); MS (APCI⁺) m/e 230 (M+H).

(2R)-2-(((4-bromophenyl)Sulfonyl)Amino)-3-(2-naphthyl)Propanehydroxamic Acid (5-n-vi)

A suspension of D-Napthylalanine hydrochloride methyl ester (1.33 g, 5 mmole), (i-Pr₂)NEt (2.61 mL, 15 mmole) and 4-bromobenzesulfonyl chloride (1.53 g, 6 mmol) in CH₂Cl₂ (50 mL) was stirred at rt overnight. The solution was washed with 5% NaHCO₃, dried (Na₂SO₄), concentrated, then chromatographed (CH₂Cl₂ to 1% MeOH/CH₂Cl₂) and concentrated to provide 2.05 g of the sulfonamide ester. This material was dissolved in 1,4-dioxane (50 mL) and 25 mL of aqueous hydroxylamine (50% w/w) was added. The mixture was allowed to stand at rt for 48 h, then concentrated onto silica, chromatographed (2% to 10% MeOH/CH₂Cl₂), the solid residue triturated with water, and dried to provide 1.45 g (64%) of 5-n-vi: R_{f} 0.35 (2% MeOH/CH₂Cl₂); ¹H NMR (DMSO-d₆) δ 9.26 (bs, 1H), 7.90-7.20 (m, 11H), 3.88 (dd, 1H), 2.90 (m, 2H); MS (electrospray-) m/e 447, 449 (M−H). Anal. Calcd for C₁₉H₁₇N₂O₄SBr.0.5H₂O: C, 49.79; H, 3.96; N, 6.11. Found: C, 49.71; H, 3.90; N, 5.97.

(2R)-3-(2-naphthyl)-2-((2-naphthylsulfonyl)Amino)Propanehydroxamic Acid (5-n-ii)

A suspension of D-Napthylalanine hydrochloride methyl ester (1.33 g, 5 mmole), (i-Pr₂)NEt (2.61 mL, 15 mmole) and 4-napthalenesulfonyl chloride (1.36 g, 6 mmol) in CH₂Cl₂ (50 mL) was stirred at rt overnight. The solution was washed with 5% NaHCO₃, dried (Na₂SO₄), concentrated, then chromatographed (CH₂Cl₂ to 1% MeOH/CH₂Cl₂) and concentrated to provide 2.02 g of the sulfonamide ester. This material was dissolved in 1,4-dioxane (50 mL) and 25 mL of aqueous hydroxylamine (50% w/w) was added. The mixture was allowed to stand at rt for 48 h, then concentrated onto silica, chromatographed (2% to 10% MeOH/CH₂Cl₂), and dried to provide 1.15 g (55%) of 5-n-ii: R_{f} 0.33 (2% MeOH/CH₂Cl₂); ¹H NMR (DMSO-d₆) δ 9.19 (bs, 2H), 8.17 (s, 1H), 7.95-7.35 (m, 12H), 7.17 (d, 1H), 3.97 (t, 1H), 2.83 (m, 2H); MS (electrospray-) m/e 419 (M+H). Anal. Calcd for C₂₃H₂₀N₂O₄S·0.75H₂O: C, 63.85; H, 4.99; N, 6.45. Found: C, 63.57; H, 4.74; N, 6.74.

### Example 14

### Antibacterial Testing

The crude compounds were screened in a representative high throughput screening assay for antibacterial activity, and compounds 5-n-ii and 5-n-vi were found to have activities minimum inhibitory concentrations (MIC's) of 0.7-1.5 μM and 3-6 μM against E. coli, respectively. This activity was verified by manual solution synthesis of analytically pure material as described in Example 6 above, which had identical activity.

### Example 15

### Functional Screening

The compounds are screened for binding affinity using MASS or conventional high-throughput functional screens. The best scoring compounds from docking a 256-member library against the 16S A-site ribosomal RNA structure are shown in the table 5 below. The DOCK scores ranged from −308.8 to −144.2 as listed in Table 5. The MASS assay was performed with the 27-mer model RNA sequence of the 16S A-site whose NMR structure has been determined. The transcription/translation assay was based on expression of a luciferase plasmid.

TABLE 5
DOCK scores correlated with mass spectrometry
and biological assay

| Compound | DOCK score | MASS KD | Activity¹ |
|---|---|---|---|
| Paromomycin | −308.8 | 0.5 μM | 0.3 μM |
| 170046 | −303.4 | >50 | >100 |
| 169999 | −299.0 | >50 | >100 |
| 169963 | −293.9 | >50 | >100 |
| 170070 | −290.2 | >50 | >100 |
| 169970 | −288.9 | 1.5 | 2.5 |
| 169961 | −288.5 | 5.0 | 10 |
| 170003 | −287.8 | >50 | >100 |
| 169995 | −286.4 | >50 | >100 |
| 169993 | −286.0 | >50 | >100 |
| 170072 | −282.6 | >50 | >100 |
| 170078 | −281.6 | 5.0 | 10 |
| 169985 | −280.1 | 4.0 | 10 |
| 169998 | −278.0 | >50 | >100 |

¹Inhibition of protein synthesis in transcription/translation assay for luciferase reporter.

Paromomycin is an aminoglycoside antibiotic known to bind to the A-site RNA structure. The NMR structure was determined with paromomycin bound at the A-site. Paromomycin had the best DOCK contact score, along with high chemical and energy scores. The docking results for these compounds have been correlated with their binding affinity for a 16S RNA fragment using MASS mass spectrometry, and their ability to inhibit protein synthesis in a transcription/translation assay. Four of the 12 compounds with the best DOCK scores had good affinity (<10 μM) for the RNA in the MASS assay and inhibited translation of a luciferase plasmid at <10 μM. In addition, all 9 of the “good” binders in the MASS assay scored in the top 30% in the DOCK calculation.

Ibis compound 169970 had the best energy score of any compound, but had a poor contact score. This result suggests that the biological activity may be increased further by modifying the structure to increase the number of close contacts with the 16S A-site RNA.

### Example 16

### Target Site of TAR

The NMR solution structure of TAR RNA (Varani, et al., J. Mol. Biol., 1995, 253, 313) has been used in the study of virtual screening for HIV-1 TAR RNA ligands. The compounds present in the Available Chemicals Database (ACD) have been partitioned into a number of subsets according to their formal charges (neutral, +1, +2, etc) and DOCKed to the TAR structure. Five aminoglycoside antibiotics were among the 20 compounds with the best binding energies.

In addition, a number of compounds were docked to TAR with subsequent evaluation of the solvation/desolvation energy. ACD 00001199 and ACD 00192509 show relatively low energies of solvation/desolvation as well as low IC₅₀ values.

### Example 17

### L11/Thiostrepton—An Example of a High Throughput RNA/Protein Assay

RNA molecules play a numerous roles in cellular functions that range from structural to enzymatic in nature. These RNA molecules may work as single large molecules, in complexes with one or more proteins, or in partnership with one or more RNA molecules. Some of these complexes, such as those found in the ribosome, have been virtually intractable as high throughput screening targets due to their immense size and complexity. The ribosome presents a particularly rich source of RNA structures and functions that would appear, at first glance, to be highly effective drug targets. A large number of natural antibiotics exist that are directed against ribosomal targets indicating the general success of this strategy. These include the aminoglycosides, kirromycin, neomycin, paromomycin, thiostrepton, and many others. Thiostrepton, a cyclic peptide based antibiotic, inhibits several reactions at the ribosomal GTPase center of the 50S ribosomal subunit. Evidence exists that thiostrepton acts by binding to the 23S rRNA component of the 50S subunit at the same site as the large ribosomal protein L11. The binding of L11 to the 23S rRNA causes a large conformation shift in the proteins tertiary structure. The binding of thiostrepton to the rRNA appears to cause an increase in the strength of the L11/23S rRNA interactions and prevents a conformational transition event in the L11 protein thereby stalling translation. Unfortunately, thiostrepton has very poor solubility, relatively high toxicity, and is not generally useful as an antibiotic. The discovery of new, novel, antibiotics directed against these types of targets would be of great value.

The design of high throughput assays to discover new antibiotics directed against ribosomal targets has been difficult, in part, due to the large structures involved and the low binding affinity of the RNA/protein interactions. Recently, a tremendous amount of data has been generated concerning RNA structures in the ribosome. This data has elucidated a number of structures and enabled the prediction of many others. Further, the use of the SPA assay format allows for assays to be run without washing or other steps that lower the concentrations of binding components. This allows one to examine binding interactions with very low (>1 μM) Kd's.

The mode of action of thiostrepton appears to be to stabilize a region of the 23S rRNA and by doing so prevent a structural transition in the L11 protein. Among the many assays that look at RNA/protein interactions, an SPA assay has been designed to look for small molecules that could be effective as thiostrepton “like” agents. This assay uses a radiolabeled small fragment of the 23S rRNA, a biotinylated 75 amino acid fragment of the L11 protein that contains the 23S rRNA binding domain and thiostrepton. The folding conditions of the secondary and tertiary structures of the 23S rRNA fragment have been examined as have the binding conditions of the L11 fragment to the 23S rRNA. The L11-thiostrepton assay has been optimized so that the 23S rRNA fragment is in an unfolded state prior to the addition of compounds. Addition of the L11 fragment to this unfolded RNA results in no detectable binding interaction. The high throughput assay is run by mixing the 23S rRNA fragment, under destabilizing conditions, with compounds of interest, incubating this mixture, and then adding the L11 fragment. Streptavidin-coated SPA beads are added for binding detection. Thiostrepton is used as a positive control. Addition of thiostrepton to the RNA promotes the correct secondary and/or tertiary folding of the structure and allows the L11 fragment to bind leading to the generation of a signal in the assay.

A tested paradigm has been developed for designing, developing and performing high and low throughput assays to look at RNA/protein function, structure, and binding in bacteria. The L11/thiostrepton assay described above is but one of a number of RNA/protein interaction and functional assays that have been designed and developed for high and low throughput screening. Others include functional assays to measure RnaseP, RnaseE, and EF-Tu activity. An assays to examine the function of the bacterial signal recognition particle and S30 assembly is also contemplated.

### Example 18

### P48-4.5S Interaction

The P48 protein-binding region of the 4.5S RNA present in the signal recognition particle of bacteria has been selected as a target. The binding of P48 to 4.5S RNA is essential for bacteria to survive, and development of an inhibitor of this binding should generate a novel; class of antimicrobial agent. Using compounds (˜2×105) from the Available Chemicals Directory (ACD), as well as from additional libraries, initial screening using DOCK (Meng, et al., J. Comp. Chem., 1992, 13, 505-524, incorporated herein by reference in its entirety) (version 4.0) can be carried out. This should leave about 15-20% of the database which have reasonably good shape complementarity in docking to the NMR structure of the 46mer, which is from the assymetric bulged regions of E. coli 4.5S RNA. A pseudobrownian Monte Carlo search in torsion angle space is performed using the program ICM (version 2.6), coupled with local minimization of each conformation, for automated flexible docking of that truncated set of potential ligands to the NMR structure and score for predicted affinity using an empirical free energy function.

Approximately 2000 of the best scoring compounds will be examined for experimental testing of their capability to inhibit the binding of P48 to 4.5S RNA. Inhibition of P48-4.5S RNA binding produced by the selected compounds will be measured using (his)₆-tagged P48 and ³³P RNA in a high-throughput scintillation proximity assay system. The structure-activity relationship among these 2000 compounds will serve as the basis for an expanded synthetic effort.

Docking of small molecules to the region of the asymmetric RNA bulges is expected to identify compounds with a high probability of selectively destabilizing the 4.5S—P48 interaction in vitro. The structure for the target RNA will be determined using NMR in the first phase of this proposal. Compounds (approaching 2×10⁵) from the Available Chemicals Directory (ACD) will be docked to the structure and scored for predicted affinity. The best molecules will be screened for their ability to disrupt the RNA-protein interaction. Quantitative structure-activity relationship (QSAR) studies will be performed on the most active compounds to identify critical features and interactions with the RNA. New compounds (˜20,000) will be prepared through combinatorial addition and/or repositioning of hydrogen bonding, aromatic, and charged functional groups to enhance the activity and specificity of the compounds for the bacterial SRP relative to the human counterpart. In addition, a pseudobrownian Monte Carlo search in torsion angle space using the program ICM2.6 (Abagyan, et al., J. Comp. Chem., 1994, 15, 488-506, incorporated herein by reference in its entirety) will be performed, coupled with local minimization of each conformation, for automated flexible docking of the truncated database to the NMR structural models.

In order to rank the ligands after flexible docking is completed, a function to estimate their binding free energies is used. There are a number of empirical methods for estimation of the free energy of binding, but empirical free energy function derived from the thermodynamic binding cycle is intended to be used (Filikov, et al., J. Comp.-Aided Molec. Design, 1998, 12, 1-12, which is incorporated herein by reference in its entirety).

### Example 19

### Inhibition of Translation of an mRNA Containing a Molecular Interaction Site by a “Small” Molecule Identified by Molecular Docking

Translation of mRNAs in eukaryotic cells follows formation of an initiation complex at the 5′-cap (m⁷ Gppp). A variety of initiation factors bind to the 5′-cap to form a pre-initiation complex before the 40S ribosomal subunit binds to the 5′-untranslated region upstream of the AUG start codon. Pain, Eur. J. Biochem., 1996, 236, 747-771. It has been demonstrated that RNA secondary structures near the 5′-cap can affect the rates of translation of mRNAs. Kozak, J. Biol. Chemistry, 1991, 266, 19867-19870. These RNA structures can bind proteins and inhibit the level of translation. Standart, et al., Biochimie, 1994, 76, 867-879. The translational machinery has an ATP-dependant RNA helicase activity associated with the eIF-4a/eIF-4b complex, and under normal conditions, the RNA structures are opened by the helicase and do not slow the rate of translation of the mRNA. The eIF-4a has a low (−μM) affinity for the pre-initiation complex.

It is believed that stabilization of mRNA structures near the 5′-cap also could be effected by specific “small” molecules, and that such binding would reduce the translational efficiency of the mRNA. To test this hypothesis, a plasmid was constructed containing the luciferase message behind a 5′-UTR containing a 27-mer RNA construct of the HIV TAR stem-loop bulge whose structure had been determined by NMR. The resulting mRNA could be expressed and capped in a wheat germ lysate translation system supplemented with T7 polymerase following addition of m⁷G to the lysate. Insertion of a 9-base leader before the TAR structure (HIVluc+9) enhanced the translational efficiency, presumably by allowing the pre-initiation complex to form. The helicase activity associated with the pre-initiation complex can transiently melt out the TAR RNA structure, and the message is translated. Addition of a 39 amino acid tat peptide to the lysate stabilized the TAR RNA structure and inhibited the expression of the luciferase protein, as expected from a specific interaction between the TAR RNA and tat.

“Small” organic molecules were then found that could inhibit the translation of the TAR-luciferase mRNA by stabilizing the TAR RNA structure. Compounds for the Available Chemicals Directory were docked to the TAR RNA structure and scored for binding energies. Among the best 25 compounds was ACD 00001199, whose structure is shown below. This compound has been shown to bind to TAR RNA with sufficient affinity to disrupt the interaction with tat peptide at a 1 μM concentration.

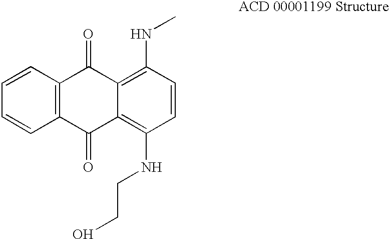

Addition of 00001199 to the wheat germ lysate translation system with the luciferase mRNA produced some inhibition of translation at very high concentrations. However, the compound was much more efficient in inhibiting translation of the luciferase mRNA containing the TAR RNA structure in the 5′-UTR, reducing translation by 50% at a 50 μM concentrations of small molecules which do not bind specifically to the TAR RNA structure did not affect translation of either mRNA construct (data not shown).

### Example 20

### Determining the Structure of a 27-mer RNA Corresponding to the 16S rRNA A Site

In order to study the structure of the 27-mer RNA corresponding to the 16S rRNA A site, of sequence 5′-GGC-GUC-ACA-CCU-UCG-GGU-GAA-GUC-GCC-3′ (SEQ ID NO:4) a chimeric RNA/DNA molecule that incorporates three deoxyadenosine (dA) residues at positions 7, 20 and 21 was prepared using standard nucleic acid synthesis protocols on an automated synthesizer. This chimeric nucleic acid of sequence 5′-GGC-GUC-dACA-CCU-UCG-GGU-GdAdA-GUC-GCC-3′ (SEQ ID NO:5) was injected as a solution in water into an electrospray mass spectrometer. Electrospray ionization of the chimeric afforded a set of multiply charged ions from which the ion corresponding to the (M-5H)⁵⁻ form of the nucleic acid was further studied by subjecting it to collisionally induced dissociation (CID). The ion was found to be cleaved by the CID to afford three fragments of m/z 1006.1, 1162.8 and 1066.2. These fragments correspond to the w₇⁽²⁻⁾, w₈⁽²⁻⁾ and the a₇-B⁽²⁻⁾ fragments respectively, that are formed by cleavage of the chimeric nucleic acid adjacent to each of the incorporated dA residues.

The observation that cleavage and fragmentation of the chimeric RNA/DNA has occurred adjacent to all three dA sites indicates that the test RNA is not ordered around the locations where the dA residues were incorporated. Therefore, the test RNA is not structured at the 7, 20 and 21 positions.

A systematic series of chimeric RNA/DNA molecules is synthesized such that a variety of molecules, each incorporating deoxy residues at different site(s) in the RNA. All such RNA/DNA members are comixed into one solution. MS analysis, as described above, are conducted on the comixture to provide a complete map or “footprint” that indicates the residues that are involved in secondary or tertiary structure and those residues that are not involved in any structure.

### Example 21

### Determining the Binding Site for Paromomycin on a 27-mer RNA Corresponding to the 16S rRNA A Site

In order to study the binding of paromomycin to the RNA of Example 20, the chimeric RNA/DNA molecule of Example 20 was synthesized using standard automated nucleic acid synthesis protocols on an automated synthesizer. A sample of this nucleic acid was then subjected to ESI followed by CID in a mass spectrometer to afford the fragmentation pattern indicating a lack of structure at the sites of dA incorporation, as described in Example 20. This indicated the accessibility of these dA sites in the structure of the chimeric nucleic acid.

Next, another sample of the chimeric nucleic acid was treated with a solution of paromomycin and the resulting mixture analyzed by ESI followed by CID using a mass spectrometer. The electrospray ionization was found to produce a set of multiply charged ions that was different from that observed for the nucleic acid alone. This was also indicative of binding of the paromomycin to the chimeric nucleic acid, because of the increased mass of the observed ion complex. Further, there was also observed, a shift in the distribution of the multiply charged ion complexes which reflected a change in the conformation of the nucleic acid in the paromomycin-nucleic acid complex into a more compact structure.

Cleavage and fragmentation of the complex by CID afforded information regarding the location of binding of the paromomycin to the chimeric nucleic acid. CID was found to produce no fragmentation at the dA sites in the nucleic acid. Thus, paromomycin must bind at or near all three dA residues. Paromomycin therefore is believed to bind to the dA bulge in this RNA/DNA chimeric target, and induces a conformational change that protects all three dA residues from being cleaved during mass spectrometry.

### Example 22

### Determining the Identity of Members of a Combinatorial Library that Bind to a Biomolecular Target

1 mL (0.6 O.D.) of a solution of a 27-mer RNA containing 3 dA residues (from Example 20) was diluted into 500 μL of 1:1 isopropanol:water and adjusted to provide a solution that was 150 mM in ammonium acetate, pH 7.4 and wherein the RNA concentration was 10 mM. To this solution was added an aliquot of a solution of paromomycin acetate to a concentration of 150 nM. This mixture was then subjected to ESI-MS and the ionization of the nucleic acid and its complex monitored in the mass spectrum. A peak corresponding to the (M-5H)⁵⁻ ion of the paromomycin-27mer complex is observed at an m/z value of 1907.6. As expected, excess 27-mer is also observed in the mass spectrum as its (M-5H)⁵⁻ peak at about 1784. The mass spectrum confirms the formation of only a 1:1 complex at 1907.6 (as would be expected from the addition of the masses of the 27-mer and paromomycin) and the absence of any bis complex that would be expected to appear at an m/z of 2036.5.

To the mixture of the 27-mer RNA/DNA chimeric and paromomycin was next added 0.7 mL of a 10 μM stock solution of a combinatorial library such that the final concentration of each member of the combinatorial library in this mixture with 27-mer target was ˜150 nM. This mixture of the 27-mer, paromomycin and combinatorial compounds was next infused into an ESI-MS at a rate of 5 mL/min. and a total of 50 scans were summed (4 microscans each), with 2 minutes of signal averaging, to afford the mass spectrum of the mixture.

The ESI mass spectrum so obtained demonstrated the presence of new signals for the (M-5H)⁵⁻ ions at m/z values of 1897.8, 1891.3 and 1884.4. Comparing these new signals to the ion peak for the 27-mer alone the observed values of m/z of those members of the combinatorial library that are binding to the target can be calculated. The masses of the binding members of the library were determined to be 566.5, 534.5 and 482.5, respectively. Knowing the structure of the scaffold, and substituents used in the generation of this library, it was possible to determine what substitution pattern (combination of substituents) was present in the binding molecules.

It was determined that the species of m/z 482.5, 534.5 and 566.5 would be the library members that bore the acetic acid+MPAC groups, the aromatic+piperidyl guanidine groups and the MPAC+guanidylethylamide groups, respectively. In this manner, if the composition of the combinatorial library is known a priori, then the identity of the binding components is straightforward to elucidate.

The use of FTMS instrumentation in such a procedure enhances both the sensitivity and the accuracy of the method. With FTMS, this method is able to significantly decrease the chemical noise observed during the electrospray mass spectrometry of these samples, thereby facilitating the detection of more binders that may be much weaker in their binding affinity. Further, using FTMS, the high resolution of the instrument provides accurate assessment of the mass of binding components of the combinatorial library and therefore direct determination of the identity of these components if the structural make up of the library is known.

### Example 23

### Determining the Site of Binding for Members of a Combinatorial Library that Bind to a Biomolecular Target

The mixture of 27-mer RNA/DNA chimeric nucleic acid, as target, with paromomycin and the combinatorial library of compounds from Example 22 was subjected to the same ESI-MS method as described in Example 22. The ESI spectrum from Example 21 showed new signals arising from the complexes formed from binding of library members to the target, at m/z values of 1897.8, 1891.3 and 1884.4. The paromomycin-27mer complex ion was observed at an n/z of 1907.3.

Two complex ions were selected from this spectrum for further resolution to determine the site of binding of their component ligands on the 27-mer RNA/DNA chimeric. First, the ions at 1907.3, that correspond to the paromomycin-27mer complex, were isolated via an ion-isolation procedure and then subjected to CID. No cleavage was found to occur and no fragmentation was observed in the mass spectrum. This indicates that the paromomycin binds at or near in the bulged region of this nucleic acid where the three dA residues are present. Paromomycin therefore protects the dA residues in the complex from fragmentation by CID.

Similarly, the ions at m/z 1897.8, that correspond to the complex of a library member with the 27-mer target, were isolated via an ion-isolation procedure and then subjected to CID using the same conditions used for the previous complex, and the data was averaged for 3 minutes. The resulting mass spectrum revealed six major fragment ions at m/z values of 1005.8, 1065.6, 1162.8, 2341.1, 2406.3 and 2446.0. The three fragments at m/z 1005.8, 1065.6 and 1162.8 correspond to the w₆⁽²⁻⁾, a₇-B⁽²⁻⁾ and w₇⁽²⁻⁾ ions from the nucleic acid target. The three ions at higher masses of 2341.1, 2406.3 and 2446.0 correspond to the a₂₀-B⁽³⁻⁾ ion +566 Da, w₂₁⁽³⁻⁾ ion +566 Da and the a₂₁-B⁽³⁻⁾ ion +566 Da. The data demonstrates at least two findings: first, since only the nucleic acid can be activated to give fragment ions in this ESI-CID experiment, the observation of new fragment ions indicates that the 1897.8 ion peak results from a library member bound to the nucleic acid target. Second, the library member has a molecular weight of 566. This library member binds to the GCUU tetraloop or the four base pairs in the stem structure of the nucleic acid target (the RNA/DNA chimeric corresponding to the 16S rRNA A site) and it does not bind to the bulged A site or the 6-base pair stem that contains the U*U mismatch pair of the nucleic acid target.

Further detail on the binding site of the library member can be gained by studying its interaction with and influence on fragmentation of target nucleic acid molecules where the positions of deoxynucleotide incorporation are different.

### Example 24

### Determining the Identity of a Member of a Combinatorial Library that Binds to a Biomolecular Target and the Location of Binding to the Target

A 10 mM solution of the 27-mer RNA target, corresponding to the 16S rRNA A-site that contains 3 dA residues (from Example 20), in 100 mM ammonium acetate at pH 7.4 was treated with a solution of paromomycin acetate and an aliquot of a DMSO solution of a second combinatorial library to be screened. The amount of paromomycin added was adjusted to afford a final concentration of 150 nM. Likewise, the amount of DMSO solution of the library that was added was adjusted so that the final concentration of each of the 216 member components of the library was ˜150 nM. The solution was infused into a Finnigan LCQ ion trap mass spectrometer and ionized by electrospray. A range of 1000-3000 m/z was scanned for ions of the nucleic acid target and its complexes generated from binding with paromomycin and members of the combinatorial library. Typically 200 scans were averaged for 5 minutes. The ions from the nucleic acid target were observed at m/z 1784.4 for the (M-5H)⁵⁻ ion and 2230.8 for the (M-4H)⁴⁻ -ion. The paromomycin-nucleic acid complex was also observed as signals of the (M-5H)⁵⁻ ion at m/z 1907.1 and the (M-4H)⁴⁻ ion at m/z 2384.4u.

Analysis of the spectrum for complexes of members of the combinatorial library and the nucleic acid target revealed several new signals that arise from the noncovalent binding of members of the library with the nucleic acid target. At least six signals for such noncovalent complexes were observed in the mass spectrum. Of these the signal at the lowest m/z value was found to be a very strong binder to the nucleic acid target. Comparison of the abundance of this ligand-nucleic acid complex ion with the abundance of the ion derived from the paromomycin-nucleic acid complex revealed a relative binding affinity (apparent KD) that was similar to that for paromomycin.

MS/MS experiments, with ˜6 minutes of signal averaging, were also performed on this complex to further establish the molecular weight of the bound ligand. A mass of 730.0±2 Da was determined, since the instrument performance was accurate only to +1.5 Da. Based on this observed mass of the bound ligand and the known structures of the scaffold and substituents used in generating the combinatorial library, the structure of the ligand was determined to bear either of three possible combinations of substituents on the PAP5 scaffold. The MS/MS analysis of this complex also revealed weak protection of the dA residues of the hybrid RNA/DNA from CID cleavage. Observation of fragments with mass increases of 730 Da showed that the molecule binds to the upper stem-loop region of the rRNA target.

### Example 25

### Determining the Identity of Members of a Combinatorial Library that Bind to a Biomolecular Target and the Location of Binding to the Target

A 10 mM solution of the 27-mer RNA target, corresponding to the 16S rRNA A-site that contains 3 dA residues (from Example 20), in 100 mM ammonium acetate at pH 7.4 was treated with a solution of paromomycin acetate and an aliquot of a DMSO solution of a third combinatorial library to be screened. The amount of paromomycin added was adjusted to afford a final concentration of 150 nM. Likewise, the amount of DMSO solution of the library that was added was adjusted so that the final concentration of each of the 216 member components of the library was ˜150 nM. The solution was infused into a Finnigan LCQ ion trap mass spectrometer and ionized by electrospray. A range of 1000-3000 m/z was scanned for ions of the nucleic acid target and its complexes generated from binding with paromomycin and members of the combinatorial library. Typically 200 scans were averaged for 5 minutes. The ions from the nucleic acid target were observed at m/z 1784.4 for the (M-5H)⁵⁻ ion and 2230.8 for the (M-4H)⁴⁻ ion. The paromomycin-nucleic acid complex was also observed as signals of the (M-5H)⁵⁻ ion at m/z 1907.1 and the (M-4H)⁴⁻ ion at m/z 2384.4 u.

Analysis of the spectrum for complexes of members of the combinatorial library and the nucleic acid target revealed several new signals that arise from the noncovalent binding of members of the library with the nucleic acid target. At least two major signals for such noncovalent complexes were observed in the mass spectrum. MS/MS experiments, with ˜6 minutes of signal averaging, were also performed on these two complexes to further establish the molecular weights of the bound ligands.

The first complex was found to arise from the binding of a molecule of mass 720.2±2 Da to the target. Two possible structures were deduced for this member of the combinatorial library based on the structure of the scaffold and substituents used to build the library. These include a structure of mass 720.4 and a structure of mass 721.1. MS/MS experiments on this ligand-target complex ion using CID demonstrated strong protection of the A residues in the bulge structure of the target. Therefore this ligand must bind strongly to the bulged dA residues of the RNA/DNA target.

The second major complex observed from the screening of this library was found to arise from the binding of a molecule of mass 665.2±2 Da to the target. Two possible structures were deduced for this member of the library based on the structure of the scaffold and substituents used to build the library. MS/MS experiments on this ligand-target complex ion using CID demonstrated strong fragmentation of the target. Therefore this ligand must not bind strongly to the bulged dA residues of the RNA/DNA target. Instead the fragmentation pattern, together with the observation of added mass bound to fragments from the loop portion of the target, suggest that this ligand must bind to residues in the loop region of the RNA/DNA target.

### Example 26

### Simultaneous Screening of a Combinatorial Library of Compounds Against Two Nucleic Acid Targets

The two RNA targets to be screened are synthesized using automated nucleic acid synthesizers. The first target (A) is the 27-mer RNA corresponding to the 16S rRNA A site and contains 3 dA residues, as in Example 20. The second target (B) is the 27-mer RNA bearing 3 dA residues, and is of identical base composition but completely scrambled sequence compared to target (A). Target (B) is modified in the last step of automated synthesis by the addition of a mass modifying tag, a polyethylene glycol (PEG) phosphoramidite to its 5′-terminus. This results in a mass increment of 3575 in target (B), which bears a mass modifying tag, compared to target (A).

A solution containing 10 mM target (A) and 10 mM mass modified target (B) is prepared by dissolving appropriate amounts of both targets into 100 mM ammonium acetate at pH 7.4. This solution is treated with a solution of paromomycin acetate and an aliquot of a DMSO solution of the combinatorial library to be screened. The amount of paromomycin added is adjusted to afford a final concentration of 150 nM. Likewise, the amount of DMSO solution of the library that is added is adjusted so that the final concentration of each of the 216 member components of the library is ˜150 nM. The library members are molecules with masses in the 700-750 Da range. The solution is infused into a Finnigan LCQ ion trap mass spectrometer and ionized by electrospray. A range of 1000-3000 m/z is scanned for ions of the nucleic acid target and its complexes generated from binding with paromomycin and members of the combinatorial library. Typically 200 scans are averaged for 5 minutes.

The ions from the nucleic acid target (A) are observed at m/z 1486.8 for the (M-6H)⁶⁻ ion, 1784.4 for the (M-5H)⁵⁻ ion and 2230.8 for the (M-4H)⁴⁻ ion. Signals from complexes of target (A) with members of the library are expected to occur with m/z values in the 1603.2-1611.6, 1924.4-1934.4 and 2405.8-2418.3 ranges.

Signals from complexes of the nucleic acid target (B), that bears a mass modifying PEG tag, with members of the combinatorial library are observed with m/z values in the 2199-2207.4, 2639-2649 and 3299-3311 ranges. Therefore, the signals of noncovalent complexes with target (B) are cleanly resolved from the signals of complexes arising from the first target (A). New signals observed in the mass spectrum are therefore readily assigned as arising from binding of a library member to either target (A) or target (B).

Extension of this mass modifying technique to larger numbers of targets via the use of unique, high molecular weight neutral and cationic polymers allows for the simultaneous screening of more than two targets against individual compounds or combinatorial libraries.

### Example 27

### Simultaneous Screening of a Combinatorial Library of Compounds Against Two Peptide Targets

The two peptide targets to be screened are synthesized using automated peptide synthesizers. The first target (A) is a 27-mer polypeptide of known sequence. The second target (B) is also a 27-mer polypeptide that is of identical amino acid composition but completely scrambled sequence compared to target (A). Target (B) is modified in the last step of automated synthesis by the addition of a mass modifying tag, a polyethylene glycol (PEG) chloroformate to its amino terminus. This results in a mass increment of ˜3600 in target (B), which bears a mass modifying tag, compared to target (A).

A solution containing 10 mM target (A) and 10 mM mass modified target (B) is prepared by dissolving appropriate amounts of both targets into 100 mM ammonium acetate at pH 7.4. This solution is treated an aliquot of a DMSO solution of the combinatorial library to be screened. The amount of DMSO solution of the library that is added is adjusted so that the final concentration of each of the 216 member components of the library is ˜150 nM. The library members are molecules with masses in the 700-750 Da range. The solution is infused into a Finnigan LCQ ion trap mass spectrometer and ionized by electrospray. A range of 1000-3000 m/z is scanned for ions of the polypeptide target and its complexes generated from binding with members of the combinatorial library. Typically 200 scans are averaged for 5 minutes.

The ions from the polypeptide target (A) and complexes of target (A) with members of the library are expected to occur at much lower m/z values that the signals from the polypeptide target (B), that bears a mass modifying PEG tag, and its complexes with members of the combinatorial library. Therefore, the signals of noncovalent complexes with target (B) are cleanly resolved from the signals of complexes arising from the first target (A). New signals observed in the mass spectrum are therefore readily assigned as arising from binding of a library member to either target (A) or target (B). In this fashion, two or more peptide targets may be readily screened for binding against an individual compound or combinatorial library.

### Example 28

### Gas-Phase Dissociation of Nucleic Acids for Determination of Structure

Nucleic acid duplexes can be transferred from solution to the gas phase as intact duplexes using electrospray ionization and detected using a Fourier transform, ion trap, quadrupole, time-of-flight, or magnetic sector mass spectrometer. The ions corresponding to a single charge state of the duplex can be isolated via resonance ejection, off-resonance excitation or similar methods known to those familiar in the art of mass spectrometry. Once isolated, these ions can be activated energetically via blackbody irradiation, infrared multiphoton dissociation, or collisional activation. This activation leads to dissociation of glycosidic bonds and the phosphate backbone, producing two series of fragment ions, called the w-series (having an intact 3′-terminus and a 5′-phosphate following internal cleavage) and the a-Base series (having an intact 5′-terminus and a 3′-furan). These product ions can be identified by measurement of their mass/charge ratio in an MS/MS experiment.

Abundances of the w and a-Base ions result from collisional activation of the (M-5H)⁵⁻ ions from a DNA:DNA duplex containing a G-G mismatch base pair. Substantial fragmentation is observed in both strands adjacent to the mismatched base pair. Following collisional activation of the control DNA:DNA duplex ion, some product ions are common, but the pattern of fragmentation differs significantly from the duplex containing the mismatched base pair. Analysis of the fragment ions and the pattern of fragmentation allows the location of the mismatched base pair to be identified unambiguously. In addition, the results suggest that the gas phase structure of the duplex DNA ion is altered by the presence of the mismatched pair in a way which facilitates fragmentation following activation.

A second series of experiments with three DNA:RNA duplexes was carried out. An A-C mismatched pair has been incorporated into the duplex. Extensive fragmentation producing w and α-Base ions is observed adjacent to the mismatched pair. However, the increased strength of the glycosidic bond in RNA limits the fragmentation of the RNA strand. Hence, the fragmentation is focussed onto the DNA strand. AC-C mismatched base pair has been incorporated into the duplex, and enhanced fragmentation is observed at the site of the mismatched pair. As above, fragmentation of the RNA strand is reduced relative to the DNA strand. The fragmentation observed for the control RNA:DNA duplex containing all complementary base pairs shows a common fragmentation pattern between the G₅—T₄ bases in all three cases. However, the extent of fragmentation is reduced in the complementary duplexes relative to the duplexes containing base pair mismatches.

### Example 29

### MASS Analysis of RNA—Ligand Complex to Determine Binding of Ligand to Molecular Interaction Site

The ability to discern through mass spectroscopy whether or not a proposed ligand binds to a molecular interaction site of an RNA can be shown. The mass spectroscopy of an RNA segment having a stem-loop structure with a ligand, schematically illustrated by an unknown, functionalized molecule was carried out. The ligand is combined with the RNA fragment under conditions selected to facilitate binding and the result in complex is analyzed by a multi target affinity/specificity screening (MASS) protocol. This preferably employs electrospray ionization Fourier transform ion cyclotron resonance mass spectrometry as described hereinbefore and in the references cited herein. “Mass chromatography” as described above permits one to focus upon one bimolecular complex and to study the fragmentation of that one complex into characteristic ions. The situs of binding of ligand to RNA can, thus, be determined through the assessment of such fragments; the presence of fragments corresponding to molecular interaction site and ligand indicating the binding of that ligand to that molecular interaction site.

AMASS analysis of a binding location for a non-A site binding molecule was carried out. The isolation through “mass chromatography” and subsequent dissociation of the (M-5H)⁵⁻ complex is observed at m/z 1919.8. The mass shift observed in select fragments relative to the fragmentation observed for the free RNA provides information about where the ligand is bound. The (2−) fragments observed below m/z 1200 correspond to the stem structure of the RNA; these fragments are not mass shifted upon Complexation. This is consistent with the ligand not binding to the stem structure.

A MASS analysis of binding location for the non-A site binding molecule was also carried out. Isolation (i.e. “mass chromatography”) and subsequent dissociation of the (M-5H)⁵⁻ complex observed at n/z 1929.4 provides significant protection from fragmentation in the vicinity of the A-site. This is evidenced by the reduced abundance of the w and a-base fragment ions in the 2300-2500 m/z range. The mass shift observed in select fragments relative to the fragmentation observed for the free RNA provides information about where the ligand is bound. The exact molecular mass of the RNA can act as an internal or intrinsic mass label for identification of molecules bound to the RNA. The (2−) fragments observed below m/z 1200 correspond to the stem structure of the RNA. These fragments are not mass shifted upon Complexation—consistent with ligand not being bound to the stem structure. Accordingly, the location of binding of ligands to the RNA can be determined.

### Example 30

### Determination of Specificity and Affinity of Ligand Libraries to RNA Targets

A preferred first step of MASS screening involves mixing the RNA target (or targets) with a combinatorial library of ligands designed to bind to a specific site on the target molecule(s). Specific noncovalent complexes formed in solution between the target(s) and any library members are transferred into the gas phase and ionized by ESI. As described herein, from the measured mass difference between the complex and the free target, the identity of the binding ligand can be determined. The dissociation constant of the complex can be determined in two ways: if a ligand with a known binding affinity for the target is available, a relative Kd can be measured by using the known ligand as an internal control and measuring the abundance of the unknown complex to the abundance of the control, alternatively, if no internal control is available, Kd's can be determined by making a series of measurements at different ligand concentrations and deriving a Kd value from the “titration” curve.

Because screening preferably employs large numbers of similar, preferably combinatorially derived, compounds, it is preferred that in addition to determining whether something from the library binds the target, it is also determined which compound(s) are the ones which bind to the target. With highly precise mass measurements, the mass identity of an unknown ligand can be constrained to a unique elemental composition. This unique mass is referred to as the compound's “intrinsic mass label.” For example, while there are a large number of elemental compositions which result in a molecular weight of approximately 615 Da, there is only one elemental composition (C₂₃H₄₅N₅O₁₄) consistent with a monoisotopic molecular weight of 615.2963012 Da. For example, the mass of a ligand (paromomycin in this example) which is noncovalently bound to the 16S A-site was determined to be 615.2969+0.0006 (mass measurement error of 1 ppm) using the free RNA as an internal mass standard. A mass measurement error of 100 ppm does not allow unambiguous compound assignment and is consistent with nearly 400 elemental compositions containing only atoms of C, H, N, and O. The isotopic distributions shown in the expanded views are primarily a result of the natural incorporation of ¹³C atoms; because high performance FTICR can easily resolve the ¹²C-¹³C mass difference, each component of the isotopic cluster can be used as an internal mass standard. Additionally, as the theoretical isotope distribution of the free RNA can be accurately simulated, mass differences can be measured between “homoisotopic” species (in this example the mass difference is measured between species containing four ¹³C atoms).

Once the identity of a binding ligand is determined, the complex is isolated in the gas phase (i.e. “mass chromatography”) and dissociated. By comparing the fragmentation patterns of the free target to that of the target complexed with a ligand, the ligand binding site can be determined. Dissociation of the complex is performed either by collisional activated dissociation (CAD) in which fragmentation is effected by high energy collisions with neutrals, or infrared multiphoton dissociation (IRMPD) in which photons from a high power IR laser cause fragmentation of the complex.

A 27-mer RNA containing the A-site of the 16S rRNA was chosen as a target for validation experiments. The aminoglycoside paromomycin is known to bind to the unpaired adenosine residues with a Kd of 200 nM and was used as an internal standard. The target was at an initial concentration of 10 mM while the paromomycin and each of the 216 library members were at an initial concentration of 150 nM. While this example was performed on a quadrupole ion trap which does not afford the high resolution or mass accuracy of the FTICR, it serves to illustrate the MASS concept. Molecular ions corresponding to the free RNA are observed at m/z 1784.4 (M-5H+)⁵⁻ and 2230.8 4 (M-4H+)⁴⁻. The signals from the RNA-paromomycin internal control are observed at m/z 1907.1 4 (M-5H+)⁵⁻ and 2384.4 4 (M-4H+)⁴⁻. In addition to the expected paromomycin complex, a number of complexes are observed corresponding to binding of library members to the target.

One member of this library (MW=675.8±1.5) forms a strong complex with the target but MS/MS studies reveal that the ligand does not offer protection of A-site fragmentation and therefore binds to the loop region. Another member of Isis 113069 having an approximate mass of 743.8±1.5 demonstrates strong binding to the target and, as evidenced by MS/MS experiments provides protection of the unpaired A residues, consistent with binding at the A-site.

The rapid and parallel nature of the MASS approach allows large numbers of compounds to be screened against multiple targets simultaneously, resulting in greatly enhanced sample throughput and information content. In a single assay requiring less than 15 minutes, MASS can screen 10 targets against a library containing over 500 components and report back which compounds bind to which targets, where they bind, and with what binding affinity.

### Example 31

### Comparison of QXP Predicted Ligand-DNA Structures to X-Ray Crystallography

The utility of QXP in the context of ligands that bind to nucleic acid targets was evaluated. The X-ray data for netropsin (aminor groove binding drug) bound to two different duplex DNA sequences (PDB ID: 261d and 195d respectively (PDB IDs are identification codes for structures deposited in the Protein Data Bank, maintained at the Research Collaboratory for Structural Bioinformatics)) and an intercalator bound to an octamer duplex (PDB ID: 2d55) were used in validation studies. Root mean square (rms) deviations between the lowest energy docked structure (with randomly disordered ligands as initial structures) and the energy minimized X-ray structure fall with in 0.6 Å in all the cases. Given that QXP method employs Monte Carlo type algorithm to search the conformational space and to make sure that the method is reliable in yielding global minimum, at least 10 QXP docking simulations were run with very different initial ligand structures. The performance of the QXP docking method can be quantified by its ability to identify the bound conformation of the ligand within 1.0 Å rms deviation from the crystallographically observed conformation. In the test cases described above, the success rate of the QXP runs is in the 80% range. The nearly linear correlation between the rms deviation from the crystal structure and the score of the docked structure indicates that the QXP method is sufficiently accurate in predicting structures of ligand-receptor complexes.

### Example 32

### Prediction of Paromomycin-RNA Complex Structure Using the QXP Method

The QXP method was used to derive an accurate structure of a bound ligand to the RNA target. The NMR structure of the bacterial 16S ribosomal A site bound to paromomycin (Fourmy et al., Science, 1996, 274, 1367; PDB ID: 1pbr) was used as the reference state. The aminoglycoside antibiotic was removed from the ligand-RNA complex. The conformation space of paromomycin was exhaustively searched using the QXP method for the lowest energy conformers. The target RNA was held rigid whereas the paromomycin was treated as fully flexible. Multiple docking searches with the randomly disrupted paromomycin as initial structures were performed. The representative lowest energy structure identified from the search (dark grey) is superimposed on the NMR structure (light grey) of the bound complex.

### Example 33

### High Precision ESI-FTICR Mass Measurement of 16S A Site RNA/Paromomycin Complex

Electrospray ionization Fourier transform ion cyclotron resonance mass spectrometry was performed on a solution containing 5 mM 16S RNA (a 27-mer construct) and 500 nM paromomycin. A 1:1 complex was observed between the paromomycin and the RNA consistent with specific aminoglycoside binding at the A-site. The insets show the measured and calculated isotope envelopes of the (M-5H+)⁵⁻ species of the free RNA and the RNA-paromomycin complex. High precision mass measurements were acquired using isotope peaks of the (M-5H+)⁵⁻ and (M-4H+)⁴⁻ charge states of the free RNA as internal mass standards and measuring the m/z difference between the free and bound RNA.

### Example 34

### Mass of 60-Member Library Against 16S A-Site RNA

FTMS spectrum was obtained from a mixture of a 16S RNA model (10 mM) and a 60-member combinatorial library. Signals from complexes are highlighted in the insert. Binding of a combinatorial library containing 60 members to the 16S RNA model have been examined under conditions where each library member was present at 5-fold excess over the RNA. Complexes between the 16S RNA and ˜5 ligands in the library were observed.

Two of the compounds in the library had a nominal mass of 398.1 Da. Their calculated molecular weights based on molecular formulas indicate that they differ in mass by 46 mDa. Accurate measurement of the molecular mass for the respective monoisotopic (all ¹²C, ¹⁴N, and ¹⁶O) (M-5H)⁵⁻ species of the complex (m/z 1863.748) and the free RNA (m/z 1784.126) allowed the mass of the ligand to be calculated as 398.110±0.009 Da.

High resolution ESI-FTICR spectrum of the library demonstrated that both library members with a nominal molecular weight of 398.1 were present in the synthesized library.

### Example 35

### Compound Identification from A 60-Member Combinatorial Library with MASS

Based on the high precision mass measurement of the complex, the mass of the binding ligand was determined to be consistent with the library member having a chemical formula of C₁₅H₁₆N₄O₂F₆ and a molecular weight of 398.117 Da. Thus, the identity of the binding ligand was unambiguously established.

### Example 36

### Elemental Composition Constraints

Use of exact mass measurements and elemental constraints can be used to determine the elemental composition of an “unknown” binding ligand. General constraints on the type and number of atoms in an unknown molecule, along with a high precision mass measurement, allow determination of a limited list of molecular formulas which are consistent with the measured mass. The elemental composition is limited to atoms of C, H, N, and O and further constrained by the elemental composition of a “known” moiety of the molecule. Based on these constraints, the enormous number of atomic combinations which result in a molecular weight of 615.2969±0.0006 are reduced to two possibilities. In addition to unambiguously identifying intended library members, this technique allows one skilled in the art to identify unintended synthetic by-products which bind to the molecular target.

### Example 37

### Determination of the MASS Kd For 16S-Paromomycin

In a direct determination of solution phase dissociation constants (Kd's) by mass spectrometry, ESI-MS measurements of a solution containing a fixed concentration of RNA at different concentrations of ligand were obtained. By measuring the ratio of bound:unbound RNA at varying ligand concentrations, the Kd was determined by 1/slope of the “titration curve”. The MS derived value of 110 nM is in good agreement with previously reported literature value of 200 nM.

### Example 38

### Multi-Target Affinity/Specificity Screening

For the determination of ligand binding site by tandem mass spectrometry, a solution containing the molecular target or targets is mixed with a library of ligands and given the opportunity to form noncovalent complexes in solution. These noncovalent complexes are mass analyzed. The noncovalent complexes are subsequently dissociated in the gas phase via IRMPD or CAD. A comparison of the fragment ions formed from dissociation of the complex with the fragment ions formed from dissociation of the free RNA reveals the ligand binding site.

### Example 39

### MASS Analysis of 27-Member Library With 16S A-Site RNA

A MASS screening of a 27 member library against a 27-mer RNA construct representing the prokaryotic 16S A-site showed that a number of compounds formed complexes with the 16S A-site.

### Example 40

### MASS Protection Assay

MS/MS of a 27-mer RNA construct representing the prokaryotic 16S A-site containing deoxyadenosine residues at the paromomycin binding site was carried oput. A first spectrum was acquired by CAD of the (M-5H)⁵⁻ ion (m/z 1783.6) from uncomplexed RNA and exhibits significant fragmentation at the deoxyadenosine residues. A second spectrum was acquired from by CAD of the (M-5H)⁵ ion of the 16S-paromomycin complex (m/z 1907.5) under identical activation energy as employed in the top spectrum. No significant fragment ions are observed in the second spectrum consistent with protection of the binding site by the ligand.

Two combinatorial libraries containing 216 tetraazacyclophanes dissolved in DMSO were mixed with a buffered solution containing 10 mM 16S RNA such that each library member was present at 100 nM. The resulting mass spectra reveal >10 complexes between 16S RNA and library members with the same nominal mass. MS-MS spectra obtained from a mixture of a 27-mer RNA construct representing the prokaryotic 16S A-site containing deoxyadenosine residues at the paromomycin binding and the 216 member combinatorial library. In the top spectrum, ions from the most abundant complex from the first library ((M-5H)⁵⁻ m/z 1919.0) were isolated and dissociated. Dissociation of this complex generates three fragment ions at n/z 1006.1, 1065.6, and 1162.4 that result from cleavage at each dA residue. More intense signals are observed at m/z 2378.9, 2443.1, and 2483.1. These ions correspond to the w₂₁^{(3-), a}₂₀-B⁽³⁻⁾, and a₂₁-B⁽³⁻⁾ fragments bound to a library member with a mass of 676.0±0.6 Da. The relative abundances of the fragment ions are similar to the pattern observed for uncomplexed RNA, but the masses of the ions from the lower stem and tetraloop are shifted by complexation with the ligand. This ligand offers little protection of the deoxyadenosine residues, and must bind to the lower stem-loop. The library did not inhibit growth of bacteria. In the bottom spectrum, dissociation of the most abundant complex from a mixture of 16S RNA and the second library having m/z 1934.3 with the same collisional energy yields few fragment ions, the predominant signals arising from intact complex and loss of neutral adenine. The reduced level of cleavage and loss of adenine for this complex is consistent with binding of the ligand at the model A site region as does paromomycin. The second library inhibits transcription/translation at 5 mM, and has an MIC of 2-20 mM against E. coli(imp-) and S. pyogenes.

### Example 41

### Neutral Mass Tag of Eukaryotic and Prokaryotic A-Sites

Secondary structures of the 27 base RNA models used in this work correspond to the 18S (eukaryotic) and 16S (prokaryotic) A-sites. The base sequences differ in seven positions (bold), the net mass difference between the two constructs is only 15.011 Da. Mass tags were covalently added to the 5′ terminus of the RNA constructs using tradition phosphoramadite coupling chemistry.

Methodology to increase the separation between the associated signals in the mass spectra was developed in view of the overlap among signals from RNAs 16S and 18S. RNA targets modified with additional uncharged functional groups conjugated to their 5′-termini were synthesized. Such a synthetic modification is referred to herein as a neutral mass tag. The shift in mass, and concomitant m/z, of a mass-tagged macromolecule moves the family of signals produced by the tagged RNA into a resolved region of the mass spectrum. ESI-FTICR spectrum of a mixture of 27-base representations of the 16S A-site with (7 mM) and without (1 mM) an 18 atom neutral mass tag attached to the 5′-terminus was carried out in the presence of 500 nM paromomycin. The ratio between unbound RNA and the RNA-paromomycin complex was equivalent for the 16S and 16S+tag RNA targets demonstrating that the neutral mass tag does not have an appreciable effect on RNA-ligand binding.

### Example 42

### Simultaneous Screening of 16S A-Site and 18S A-Site Model RNAs Against Aminoglycoside Mixture

Paromomycin, lividomycin (MW=761.354 Da), sisomicin (MW=447.269 Da), tobramycin (MW=467.2591 Da), and bekanamycin (MW=483.254 Da) were obtained from Sigma (St. Louis, Mo.) and ICN (Costa Mesa, Calif.) and were dissolved to generate 10 mM stock solutions. 2′ methoxy analogs of RNA constructs representing the prokaryotic (16S) rRNA and eukaryotic (18S) rRNA A-site were synthesized in house and precipitated twice from 1 M ammonium acetate following deprotection with ammonia (pH 8.5). The mass-tagged constructs contained an 18-atom mass tag (C₁₂H₂₅0₉) attached to the 5′-terminus of the RNA oligomer through a phosphodiester linkage.

All mass spectrometry experiments were performed using an Apex II 70e electrospray ionization Fourier transform ion cyclotron resonance mass spectrometer (Bruker Daltonics, Billerica) employing an actively shielded 7 tesla superconducting magnet. RNA solutions were prepared in 50 mM NH₄OAc (pH 7), mixed 1:1 v:v with isopropanol to aid desolvation, and infused at a rate of 1.5 mL/min using a syringe pump. Ions were formed in a modified electrospray source (Analytica, Branford) employing an off axis, grounded electrospray probe positioned ca. 1.5 cm from the metalized terminus of the glass desolvation capillary biased at 5000 V. A counter-current flow of dry oxygen gas heated to 225C was employed to assist in the desolvation process. Ions were accumulated in an external ion reservoir comprised of an RF-only hexapole, a skimmer cone, and an auxiliary electrode for 1000 ms prior to transfer into the trapped ion cell for mass analysis. Each spectrum was the result of the coaddition of 16 transients comprised of 256 datapoints acquired over a 90,909 kHz bandwidth resulting in a 700 ms detection interval. All aspects of pulse sequence control, data acquisition, and post acquisition processing were performed using a Bruker Daltonics datastation running XMASS version 4.0 on a Silicon Graphics (San Jose, Calif.) R5000 computer.

Mass spectrometry experiments were performed in order to detect complex formation between a library containing five aminoglycosides (Sisomicin (Sis), Tobramycin (Tob), Bekanomycin (Bek), Paromomycin (PM), and Livodomycin (LV)) and two RNA targets simultaneously. Signals from the (M-5H+)⁵⁻ charge states of free 16S and 18S RNAs are detected at m/z 1801.515 and 1868.338, respectively. The mass spectrometric assay reproduces the known solution binding properties of aminoglycosides to the 16S A site model and an 18S A site model with a neutral mass linker. Consistent with the higher binding affinity of theses aminoglycosides for the 16S A-site relative to the 18S A-site, aminoglycoside complexes are observed only with the 16S rRNA target. Note the absence of 18S-paromomycin and 18S-lividomycin complexes, which would be observed at the n/z's indicated by the arrows. The inset demonstrates the isotopic resolution of the complexes. Using multiple isotope peaks of the (M-5H+)⁵⁻ and (M-4H+)⁴⁻ charge states of the free RNA as internal mass standards, the average mass measurement error of the complexes is 2.1 ppm. High affinity complexes were detected between the 16S A site 27mer RNA and paromomycin and lividomycin, respectively. Weaker complexes were observed with sisomycin, tobramycin and bekamycin. No complexes were observed between any of the aminoglycosides and the 18S A site model. Thus, this result validates the mass spectrometric assay for identifying compounds that will bind specifically to the target RNAs. No other type of high throughput assay can provide information on the specificity of binding for a compound to two RNA targets simultaneously. The binding of lividomycin to the 16S A site had been inferred from previous biochemical experiments. The mass spectrometer has been used herein to measure a KD of 28 nM for lividomycin and 110 nM for paromomycin to the 16S A site 27mer. The solution KD for paromomycin has been estimated to be between 180 nM and 300 nM.

### Example 43

### Targeted Site-Specific Gas-Phase Cleavage of Oligoribonucleotides—Application in Mass Spectrometry-Based Identification of Ligand Binding Sites

Fragmentation of oligonucleotides is a complex process, but appears related to the relative strengths of the glycosidic bonds. This observation is exploited by incorporating deoxynucleotides selectively into a chimeric 2′-O-methylribonucleotide model of the bacterial rRNA A site region. Miyaguchi, et al., Nucl. Acids Res., 1996, 24, 3700-3706; Fourmy, et al., Science, 1996, 274, 1367-1371; and Fourmy, et al., J. Mol. Biol., 1998, 277, 333-345. During CAD, fragmentation is directed to the more labile deoxynucleotide sites. The resulting CAD mass spectrum contains a small subset of readily assigned complementary fragment ions. Binding of ligands near the deoxyadenosine residues inhibits the CAD process, while complexation at remote sites does not affect dissociation and merely shifts the masses of specific fragment ions. These methods are used to identify compounds from a combinatorial library that preferentially bind to the RNA model of the A site region.

The 27-mer model of a segment of the bacterial A site region has been prepared as a full ribonucleotide, and as a chimeric 2′-O-methylribonucleotide containing three deoxyadenosine residues. RNAs R and C have been prepared using conventional phosphoramidite chemistry on solid support. Phosphoramidites were purchased from Glen Research and used as 0.1 M solutions in acetonitrile. RNA R was prepared following the procedure given in Wincott, et al., Nucl. Acids Res., 1995, 23, 2677-2684, the disclosure of which is incorporated herein by reference in its entirety. RNA C was prepared using standard coupling cycles, deprotected, and precipitated from 10 M NH₄OAc. The aminoglycoside paromomycin binds to both R and C with kD values of 0.25 and 0.45 micromolar, respectively. The reported kD values are around 0.2 μM. Recht, et al., J. Mol. Biol., 1996, 262, 421-436, Wong, et al., Chem. Biol., 1998, 5, 397-406, and Wang, et al., Biochemistry, 1997, 36, 768-779. Paromomycin has been shown previously to bind in the major groove of the 27-mer model RNA and induce a conformational change, with contacts to A1408, G1494, and G1491. Miyaguchi, et al., Nucl. Acids Res., 1996, 24, 3700-3706; Fourmy, et al., Science, 1996, 274, 1367-1371; and Fourmy, et al., J. Mol. Biol., 1998, 277, 333-345.

The mass spectrum obtained from a 5 μM solution of C mixed with 125 nM paromomycin contains [M-5H]⁵⁻ ions from free C at m/z 1783.6 and the [M-5H]⁵⁻ ions of the paromomycin-C complex at m/z 1907.3. Mass spectrometry experiments have been performed on an LCQ quadrupole ion trap mass spectrometer (Finnigan; San Jose, Calif.) operating in the negative ionization mode. RNA and ligand were dissolved in a 150 mM ammonium acetate buffer at pH 7.0 with isopropyl alcohol added (1:1 v:v) to assist the desolvation process. Parent ions have been isolated with a 1.5 m/z window, and the AC voltage applied to the end caps was increased until about 70% of the parent ion dissociates. The electrospray needle voltage was adjusted to −3.5 kV, and spray was stabilized with a gas pressure of 50 psi (60:40 N₂:O₂). The capillary interface was heated to a temperature of 180C. The He gas pressure in the ion trap was 1 mTorr. In MS-MS experiments, ions within a 1.5 Da window having the desired m/z were selected via resonance ejection and stored with q) 0.2. The excitation RF voltage was applied to the end caps for 30 ms and increased manually to 1.1 Vpp to minimize the intensity of the parent ion and to generate the highest abundance of fragment ions. A total of 128 scans were summed over n/z 700-2700 following trapping for 100 ms. Signals from the [M-4H]⁴⁻ ions of C and the complex are detected at m/z 2229.8 and 2384.4, respectively. No signals are observed from more highly charged ions as observed for samples denatured with tripropylamine. In analogy with studies of native and denatured proteins, this is consistent with a more compact structure for C and the paromomycin complex. A CAD mass spectrum obtained from the [M-5H]⁵⁻ ion of C was obtained. Fragment ions are detected at m/z 1005.6 (w6)2−, 1065.8 (a7-B)2−, 1162.6 (w7)2−, 1756.5 (M-Ad)5−, 2108.9 (w21-Ad)3−, 2153.4 (a20-B)3−, 2217.8 (w21)3−, and 2258.3 (a21-B)3−. McLuckey, et al., J. Am. Soc. Mass Spectrum., 1992, 3,60-70 and McLuckey, et al., J. Am. Chem. Soc., 1993, 115, 12085-12095. These fragment ions all result from loss of adenine from the three deoxyadenosine nucleotides, followed by cleavage of the 3′-C—O sugar bonds. In a CAD mass spectrum for the [M-5H]⁵⁻ ion of the complex between C and paromomycin obtained with the same activation energy, no fragment ions are detected from strand cleavage at the deoxyadenosine sites using identical dissociation conditions. The change in fragmentation pattern observed upon binding of paromomycin is consistent with a change in the local charge distribution, conformation, or mobility of A1492, A1493, and A1408 that precludes collisional activation and dissociation of the nucleotide.

Two combinatorial libraries containing 216 tetraazacyclophanes dissolved in DMSO were mixed with a buffered solution containing 10 μM C such that each library member is present at 100 nM. The resulting mass spectra reveal >10 complexes between C and library members with the same nominal mass. Ions from the most abundant complex from the first library ((M-5H)⁵⁻ m/z 1919.0) were isolated and dissociated. Dissociation of this complex generates three fragment ions at m/z 1006.1, 1065.6, and 1162.4 that result from cleavage at each dA residue. More intense signals are observed at m/z 2378.9, 2443,1, and 2483.1. These ions correspond to the w²¹⁽³⁻⁾, α₂₀-B⁽³⁻⁾, and α₂₁-B⁽³⁻⁾ fragments bound to a library member with a mass of 676.0=0.6Da. The relative abundances of the fragment ions are similar to the pattern observed for uncomplexed C, but the masses of the ions from the lower stem and tetraloop are shifted by complexation with the ligand. This ligand offers little protection of the deoxyadenosine residues, and must bind to the lower stem-loop. The libraries have been synthesized from a mixture of charged and aromatic functional groups, and are described as libraries 25 and 23 in: An et al., Bioorg. Med. Chem. Lett., 1998, in press. Dissociation of the most abundant complex from a mixture of C and the second library having m/z 1934.3 with the same collisional energy yields few fragment ions, the predominant signals arising from intact complex and loss of neutral adenine. The mass of the ligand (753.5 Da) is consistent with six possible compounds in the library having two combinations of functional groups. The reduced level of cleavage and loss of adenine from this complex is consistent with binding of the ligand at the model A site region as does paromomycin. The second library inhibits transcription/translation at 5 μm, and has an MIC of 2-20 μM against E. coli (imp-) and S. pyogenes.

Mass spectrometry-based assays provide many advantages for identification of complexes between RNA and small molecules. All constituents in the assay mixture carry an intrinsic mass label, and no additional modifications with radioactive or fluorescent tags are required to detect the formation of complexes. The chemical composition of the ligand can be ascertained from the measured molecular mass of the complex, allowing rapid deconvolution of libraries to identify leads against an RNA target. Incorporation of deoxynucleotides into a chimeric oligoribonucleotide generates a series of labile sites where collisionally-activated dissociation is favored. Binding of ligands at the labile sites affords protection from CAD observed in MS-MS experiments. This mass spectrometry-based protection methods of the invention can be used to establish the binding sites for small molecule ligands without the need for additional chemical reagents or radiobabeling of the RNA. The methodology can also be used in DNA sequencing and identification of genomic defects.

In accordance with preferred embodiments of the present invention, enhanced accuracy of determination of binding between target biomolecules and putative ligands is desired. It has been found that certain mass spectrometric techniques can give rise to such enhancement. As will be appreciated, the target biomolecule will always be present in excess in samples to be spectroscopically analyzed. The exact composition of such target will, similarly, be known. Accordingly, the isotopic abundances of the parent (and other) ions deriving from the target will be known to precision.

In accordance with preferred embodiments, mass spectrometric data is collected from a sample comprising target biomolecule (or biomolecules) which has been contacted with one or more, preferably a mixture of putative or trial ligands. Such a mixture of compounds may be quite complex as discussed elsewhere herein. The resulting mass spectrum will be complex as well, however, the signals representative of the target biomolecule(s) will be easily identified. It is preferred that the isotopic peaks for the target molecule be identified and used to internally calibrate the mass spectrometric data thus, collected since the M/e for such peaks is known with precision. As a result, it becomes possible to determine the exact mass shift (with respect to the target signal) of peaks which represent complexes between the target and ligands bound to it. Given the exact mass shifts, the exact molecular weights of said ligands may be determined. It is preferred that the exact molecular weights (usually to several decimal points of accuracy) be used to determine the identity of the ligands which have actually bound to the target.

In accordance with other preferred embodiments, the information collected can be placed into a relational or other database, from which further information concerning ligand binding to the target biomolecule can be extracted. This is especially true when the binding affinities of the compounds found to bind to the target are determined and included in the database. Compounds having relatively high binding affinities can be selected based upon such information contained in the database.

It is preferred that such data collection and database manipulation be achieved through a general purpose digital computer. An exemplary software program has been created and used to identify the small molecules bound to an RNA target, calculate the binding constant, and write the results to a relational database. The program uses as input a file that lists the elemental formulas of the RNA and the small molecules which are present in the mixture under study, and their concentrations in the solution. The program first calculates the expected isotopic peak distribution for the most abundant charge state of each possible complex, then opens the raw FTMS results file. The program performs a fast Fourier transform of the raw data, calibrates the mass axis, and integrates the signals in the resulting spectrum. The peaks in the spectrum are preferably identified via centroiding, are integrated, and preferably stored in a database. The expected and observed peaks are correlated, and the integrals converted into binding constants based on the intensity of an internal standard. The compound identity and binding constant data are written to a relational database. This approach allows large amounts of data that are generated by the mass spectrometer to be analyzed without human intervention, which results in a significant savings in time.

Electrospray ionization Fourier transform ion cyclotron resonance mass spectrometry of a solution which is 5 mM in 16S RNA (Ibis 16628) and 500 nM in the ligand Ibis10019 was performed. The raw time-domain dataset is automatically apodized and zerofilled twice prior to Fourier transformation. The spectrum is automatically post-calibrated using multiple isotope peaks of the (M-5H+)⁵⁻ and (M-4H+)⁴⁻ charge states of the free RNA as internal mass standards and measuring the m/z difference between the free and bound RNA. The isotope distribution of the free RNA is calculated a priori and the measured distribution is fit to the calculated distribution to ensure that m/z differences are measured between homoisotopic species (e.g. monoisotopic peaks or isotope peaks containing 4 ¹³C atoms).

Isotope clusters observed in the m/z range where RNA-ligand complexes are expected are further analyzed by peak centroiding and integration. Data was tabulated and stored in a relational database. Peaks which correspond to complexes between the RNA target and ligands are assigned and recorded in the database. If an internal affinity standard is employed, a relative Kd is automatically calculated from the relative abundance of the standard complex and the unknown complex and recorded in the database.

When computer controlled collection of the foregoing information is provided and computer control of relational databases is employed, the present invention is capable of very high throughput analysis of mass spectrometric binding information. Such control facilitates the identification of ligands having high binding affinities for the target biomolecules. Thus, automation permits the automatic calculation of the mass of the binding ligand or ligands, especially when the mass of the target is used for internal calibration purposes. From the precise mass of the binding ligands, their identity may be determined in an automated way. The dissociation constant for the ligand—target interaction may also be ascertained using either known Kd and abundance of a reference complex or by titration with multiple measurements at different target/ligand ratios. Further, tandem mass spectrometric analyses may be performed in an automated fashion such that the site of the small molecule, ligand, interaction with the target can be ascertained through fragmentation analysis. Computer input and output from the relational database is, of course, preferred.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

What is claimed is:

1. An RNA comprising a joined sequence of at least twenty-four nucleotides but not more than seventy nucleotides and having secondary structure defined by:
three nucleotides forming a first side of a first double stranded region;
two nucleotides forming a first side of an internal loop region;
four nucleotides forming a first side of a second double stranded region;
four or five nucleotides forming an end loop region;
four nucleotides forming a second side of said second double stranded region;
four nucleotides forming a second side of said internal loop region; and
three nucleotides forming a second side of said first double stranded region.

2. The RNA of claim 1 wherein said two nucleotides forming said first side of said internal loop region are of the sequence NC.

3. The RNA of claim 1 wherein said four nucleotides forming said first side of said second double stranded region are of the sequence NNNN and said four nucleotides forming said second side of said second double stranded region are of the sequence NANN.

4. The RNA of claim 1 wherein said four or five nucleotides forming said end loop region are of the sequence NNNUN or NNUN.

5. The RNA of claim 1 comprising a portion of vimentin RNA.

6. The RNA of claim 1 comprising a portion of the 3′-UTR of vimentin mRNA.

7. A purified and isolated RNA comprising a joined sequence of nucleotides having secondary structure defined by:
three nucleotides forming a first side of a first double stranded region;
two nucleotides forming a first side of an internal loop region;
four nucleotides forming a first side of a second double stranded region;
four or five nucleotides forming an end loop region;
four nucleotides forming a second side of said second double stranded region;
four nucleotides forming a second side of said internal loop region; and
three nucleotides forming a second side of said first double stranded region.

8. The RNA of claim 7 wherein said two nucleotides forming said first side of said internal loop region are of the sequence NC.

9. The RNA of claim 7 wherein said four nucleotides forming said first side of said second double stranded region are of the sequence NNNN and said four nucleotides forming said second side of said second double stranded region are of the sequence NANN.

10. The RNA of claim 7 wherein said four or five nucleotides forming said end loop region are of the sequence NNNUN or NNUN.

11. The RNA of claim 7 comprising a portion of vimentin RNA.

12. The RNA of claim 7 comprising a portion of the 3′-UTR of vimentin mRNA.

13. An in silico RNA comprising a joined sequence of nucleotides having secondary structure defined by:
three nucleotides forming a first side of a first double stranded region;
two nucleotides forming a first side of an internal loop region;
four nucleotides forming a first side of a second double stranded region;
four or five nucleotides forming an end loop region;
four nucleotides forming a second side of said second double stranded region;
four nucleotides forming a second side of said internal loop region; and
three nucleotides forming a second side of said first double stranded region.

14. The RNA of claim 13 wherein said two nucleotides forming said first side of said internal loop region are of the sequence NC.

15. The RNA of claim 13 wherein said four nucleotides forming said first side of said second double stranded region are of the sequence NNNN and said four nucleotides forming said second side of said second double stranded region are of the sequence NANN.

16. The RNA of claim 13 wherein said four or five nucleotides forming said end loop region are of the sequence NNNUN or NNUN.

17. The RNA of claim 13 comprising a portion of vimentin RNA.

18. The RNA of claim 13 comprising a portion of the 3′-UTR of vimentin mRNA.

19. An isolated RNA fragment comprising the consensus sequence 5′-NNNNCNNNNNNNUNNANNNNNNNN-3′ (SEQ ID NO:1) or 5′-NNNNCNNNNNNNUNN ANNNNNNNN-3′ (SEQ ID NO:2), wherein said sequence has a first double stranded region, an internal loop region, a second double stranded region and an end loop region, wherein each of said double stranded and internal loop regions comprises first and second sides, each of said first sides occurring 5′ to said end loop region in said consensus sequence and each of said second sides occurring 3′ to said end loop region in said consensus sequence, and wherein said first and second sides of said internal loop region are unhybridized.

20. A computer-readable medium encoded with a data structure comprising a representation of an RNA fragment having at least 60% homology across at least two species of organisms comprising the consensus sequence 5′-NNNNCNNNNNNNUNNANNNNNNNN-3′ (SEQ ID NO:1) or 5′-NNNNCNNNNNNNUNNANNNNNNNN-3′ (SEQ ID NO:2) and wherein said sequence has a first double stranded region, an internal loop region, a second double stranded region and an end loop region, wherein each of said double stranded and internal loop regions comprises first and second sides, each of said first sides occurring 5′ to said end loop region in said consensus sequence and each of said second sides occurring 3′ to said end loop region in said consensus sequence.

21. A purified and isolated RNA fragment that is conserved across at least two species comprising the sequence NNNNCNNNNNN(or absent)NUNNANNNNNNNN.

22. A purified and isolated RNA fragment comprising the human sequence UUUACAACAUAAUCUAGUUUACAGAAAAAUC.

23. An in silico representation of an RNA fragment comprising the human sequence UUUACAACAUAAUCUAGUUUACAGAAAAAUC.

24. The RNA fragment of claim 19 wherein said RNA fragment comprises up to seventy nucleotides.

25. The RNA fragment of claim 19 wherein said first side of said internal loop region consists of two nucleotides.

26. The RNA fragment of claim 25 wherein said first side of said internal loop region consists of NC.

27. The RNA fragment of claim 19 wherein said first and second sides of said second double stranded region each consist of four nucleotides.

28. The RNA fragment of claim 27 wherein said first side of said second double stranded region consists of NNNN and said second side of said second double stranded region consists of NANN.

29. The RNA fragment of claim 19 wherein said end loop region consists of four or five nucleotides.

30. The RNA fragment of claim 29 wherein said end loop region consists of NNNUN or NNUN.

31. The RNA fragment of claim 19 wherein three nucleotides form said first side of said first double stranded region, two nucleotides form said first side of said internal loop region, four nucleotides form said first side of said second double stranded region, four or five nucleotides form said end loop region, four nucleotides form said second side of said second double stranded region, four nucleotides form said second side of said internal loop region, and three nucleotides form said second side of said first double stranded region.

32. The RNA fragment of claim 31 wherein the two nucleotides forming said first side of said internal loop region are NC.

33. The RNA fragment of claim 31 wherein the four nucleotides forming said first side of said second double stranded region are NNNN and the four nucleotides forming said second side of said second double stranded region are NANN.

34. The RNA fragment of claim 31 wherein the four or five nucleotides forming the end loop region are NNNUN or NNUN.

35. The RNA fragment of claim 31 wherein said RNA fragment comprises a portion of vimentin RNA.

36. The RNA fragment of claim 35 wherein said RNA fragment comprises a portion of the 3′-UTR of vimentin RNA.

37. The RNA fragment of claim 19 comprising 5′-UUUACAACAUAAUCUAGUUUACA GAAAAAUC-3′(SEQ ID NO:2).

38. The RNA fragment of claim 31 comprising 5′-UUUACAACAUAAUCUAGUUUACA GAAAAAUC-3′(SEQ ID NO:2).


SEQUENCE LISTING (5 sequences)

SEQ ID NO: 1 - RNA, 24 nt, Artificial Sequence
  Description of Artificial Sequence Novel Sequence
nnnncnnnnnnnunnannnnnnnn

SEQ ID NO: 2 - RNA, 23 nt, Artificial Sequence
  misc_feature: Novel Sequence
nnnncnnnnnnunnannnnnnnn

SEQ ID NO: 3 - RNA, 31 nt, Artificial Sequence
  Description of Artificial Sequence Novel Sequence
uuuacaacauaaucuaguuuacagaaaaauc

SEQ ID NO: 4 - RNA, 27 nt, Artificial Sequence
  Description of Artificial Sequence Novel Sequence
ggcgucacaccuucgggugaagucgcc

SEQ ID NO: 5 - DNA, 27 bp, Artificial Sequence
  Description of Combined DNA/RNA Molecule chimeric nucleic acid
ggcgucacaccuucgggugaagucgcc